(19) United States
(12) Patent Application Publication
(10) Pub. No.: US 2003/0165949 A1
(43) Pub. Date: Sep. 4, 2003

(54) Genes abnormally expressed in myeloid leukemia cells with an MLL-AF9 fusion

(76) Inventors: San Ming Wang, Chicago, IL (US); Sanggyu Lee, Chicago, IL (US); Jianjun Chen, Chicago, IL (US); Guolin Zhou, Chicago, IL (US); Janet D. Rowley, Chicago, IL (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/329,465

(22) Filed: Dec. 23, 2002

Related U.S. Application Data
(60) Provisional application No. 60/343,826, filed on Dec. 27, 2001.

(51) Int. Cl. C12Q 1/68; C12M 1/34

(52) U.S. Cl. 435/6; 435/287.2

(57) ABSTRACT

The invention provides materials and methods for the analysis of gene expression in hematopoietic cells, such as hematopoietic stem/precursor cells of mammals. Materials of the invention include microarrays of polynucleotides collectively characterizing gene expression in hematopoietic cells, as well as a vector comprising at least one such polynucleotide and a host cell containing such a vector. Methods of the invention include diagnostic methods for identifying or detecting expression products collectively characteristic of normal, or abnormal, hematopoietic cells. Other methods of the invention include therapeutic methods for the treatment or prevention of an abnormality such as a disease or condition of a hematopoietic cell, comprising delivery of an effective amount of a suitable material, such as a pharmaceutical composition of the invention, to a cell or patient in need thereof.

### FIELD OF THE INVENTION

The invention generally is related to leukemia. More specifically, the invention is directed to materials and methods for determining gene expression in leukemic cells. In particular, the invention provides genes abnormally expressed in leukemic cells with an MLL-AF9 fusion.

### BACKGROUND

Chromosomal rearrangements are consistently associated with acute myeloid leukemias (AMLs). The generation of chimeric genes and fusion proteins via chromosomal translocation events frequently results in the deregulation of myeloid differentiation since one of the two affected genes is often a transcription factor involved in the regulation of hematopoietic differentiation.

The MLL (mixed-lineage leukemia) gene is an important regulatory gene located on chromosome band 11q23. The MLL gene is frequently involved in de novo and treatment-related leukemia, in reciprocal chromosome translocations with other genes, which results in breaks in MLL and the partner genes, and the formation of new fusion genes. Nearly 40 different partner genes have been shown to be involved in this translocation (Ziemin-van der Poel, et al., Proc. Natl. Acad. Sci., 88(23), 10735-10739, 1991; Gu, et al., Cell, 71, 701-708, 1992). The MLL-AF9 fusion, which results from a (9;11) chromosome translocation, is one of the most common examples. The disruption of the wild-type genes and formation of the fusion genes will likely alter the normal expression pattern of other genes. Abnormally expressed genes, both those whose expression is increased, as well as those whose expression is decreased, likely change the cell behavior, and eventually lead to the development of leukemia. At present, there is a need to identify the abnormally expressed genes that are unique to the t(9;11).

The current understanding of the relationship between MLL translocations and leukemia is based mainly on the cytogenetic identification, molecular cloning of partner genes, fusion gene knock-in (Corral, et al., Cell, 85(6), 853-861, 1996) and knock-out experiments. Even though this information provides strong support for a causal relationship between MLL translocations and the development of leukemia, the mechanisms of how these fusion genes cause leukemia remain unclear. One of the possibilities is that the fusion genes lead to abnormal expression of target genes. Both wild-type MLL and AF9 have transcriptional regulatory domains that suggest that these genes regulate the expression of their target genes. The new MLL/AF9 fusion gene resulting from the t(9;11) translocation contains many of the original active domains of its two components. However, it is very likely that the new fusion gene either gains a different regulatory function, or that MLL and AF9 have lost some of their original activity. These changes may affect the expression pattern of the original target genes, and may also influence the expression of many other genes. These changes will disturb the normal cell behavior, and eventually may lead to leukemia.

To date, there is very little information available about the events at the genome level to provide insights into the mechanism of leukemia development associated with any particular translocation. This lack of information of global gene expression hinders the efforts to reach a deeper understanding of the mechanisms leading to leukemia. Therefore, identification of abnormally expressed genes becomes the first step in understanding the relationship between MLL/AF9 translocation and leukemia development. The identification of genes that are specifically altered in leukemia will provide important clues to understanding the mechanism of the development of the leukemia. It will also lead to many applications, including the identification of markers for early diagnosis, targets for drug design, and indicators for treatment responsiveness and prognosis.

SAGE is a technique designed to take advantage of high-throughput sequencing technology to obtain a quantitative profile of cellular gene expression for genome-wide level. Essentially, the SAGE technique measures not the expression level of the gene, but quantifies a “tag” which represents the transcription product of the gene. The data product of the SAGE technique is a list of tags, with their corresponding count values, and thus is an unbiased digital representation of cellular gene expression. Application of the SAGE technique has generated large amounts of gene expression information from various biological systems that is not biased by the use only of known genes and ESTs.

However, there are two problems when applying the SAGE tag sequence for gene identification. The first one is that many SAGE tags identified have no match to known sequences in the database. These tags may represent potentially novel genes. It is difficult to use this tag's information for further characterization of the corresponding genes because of their short length. The second one is that many SAGE tag sequences have multiple matches with sequences in the database. These matched sequences have no similarity to each other except that they share the same SAGE tag sequence. This feature makes it difficult to determine the correct sequence corresponding to a SAGE tag among all of the matched sequences.

### SUMMARY OF THE INVENTION

The invention generally relates to materials and methods for the identification of genes abnormally expressed in leukemic cells, particularly leukemic cells having an MLL-AF9 fusion. The invention not only facilitates a qualitative assessment of gene expression in such cells, it also provides a quantitative measure of that gene expression. Moreover, these qualitative and quantitative measures of gene expression are provided on a global basis insofar as the entire cellular gene expression may be simultaneously monitored, thereby providing a global assessment of gene expression in a particular cell type or mixture of cell types. These measures beneficially provide assurance that the methods of cell identification of the invention, relying on qualitative and/or quantitative measures of cellular gene expression, leads to greater accuracy in that reliance is not confined to expression measures of one or a few putatively characteristic genes of a cell of interest. In providing both qualitative and quantitative global measures of cellular gene expression, the invention provides methods capable of fine distinctions in assessing the physiological states of cells. Accordingly, the invention provides diagnostic and therapeutic methods for assessing and/or treating leukemic cells with heretofore unknown accuracy and sensitivity.

In one aspect of the invention, materials are provided in the form of a microarray for measuring gene expression characteristic of leukemic cells comprising at least 5 polynucleotides, and preferably at least 9 polynucleotides, having distinct sequences selected from the group consisting of SEQ ID NOS: 1-244. Other microarrays of the invention comprise between 10 and 244 polynucleotides, wherein each polynucleotide has a distinct sequence selected from the group consisting of SEQ ID NOS: 1-244. Of course, such microarrays may also have duplicate polynucleotides and additional polynucleotides, e.g., control polynucleotides for use in hybridization-based assays using the microarray.

In a related aspect, the invention provides a vector comprising one or more of the novel polynucleotides described above. Any vector known in the art may be used. Preferred vectors are expression vectors functional in mammalian cells, and preferably human cells. Such vectors are useful in methods for treating leukemic conditions. In addition to vectors, the invention contemplates host cells containing such vectors or the above-described polynucleotides of the invention. A preferred host cell is a leukemic cell. A particularly preferred host cell is a human leukemic cell with an MLL-AF9 fusion.

In still another aspect, the invention provides a pharmaceutical composition comprising any one or more of the above-described materials of the invention, such as a polynucleotide, vector or host cell, in admixture with a suitable adjuvant, excipient, diluent, or carrier known in the art.

Another aspect of the invention is drawn to methods of diagnosing conditions associated with leukemia. In particular, this aspect of the invention provides a method of diagnosing a condition associated with leukemia comprising the following steps: (a) contacting a cell sample nucleic acid with a microarray as described above under conditions suitable for hybridization; (b) providing hybridization conditions suitable for hybrid formation between the cell sample nucleic acid and a polynucleotide of the microarray; (c) detecting the hybridization; and (d) diagnosing a hematopoietic condition based on the results of detecting the hybridization. Suitable hybridization conditions are those conditions that allow the detection of gene expression from identifiable expression units such as genes. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

The invention also extends to a method of treating a condition associated with leukemia comprising the following steps: (a) preparing a polynucleotide having a sequence selected from the group consisting of SEQ ID NOS: 1-244 in an amount effective to treat a condition associated with leukemia; and (b) delivering the polynucleotide to a mammal in need of such treatment. A preferred mammal in need of treatment is a human. Any condition associated with leukemia is contemplated as being amenable to the treatment methods of the invention. The treatment methods of the invention also embrace methods of preventing the onset or development of a leukemic condition such as a disease and, therefore, the mammal in need of treatment may be a mammal not exhibiting symptoms or known to be suffering from the leukemic condition (i.e., a relatively healthy mammal). Further contemplated in the treatment methods of the invention is delivery of a pharmaceutical composition comprising a polynucleotide, vector or host cell as described above.

Numerous other aspects and advantages of the invention will be apparent upon consideration of the following drawing and detailed description.

### BRIEF DESCRIPTION OF THE DRAWING

The following drawing forms part of the present specification and is included to further illustrate aspects of the invention. The invention may be better understood by reference to the drawing in combination with the detailed description of the specific embodiments presented herein.

FIG. 1. Schematic illustration of the GLGI process.

### DETAILED DESCRIPTION OF THE PREFERRED EMBODIMENTS

Ex vivo expanded hematopoietic stem cells have been used in various clinical trials and hematopoietic stem cells likely will be useful in treating or ameliorating numerous diseases. Despite this exciting potential, the mechanisms controlling gene expression in these cells and, indeed, the mechanisms by which hematopoietic stem cells differentiate, are not yet understood.

Myeloid cells originate from stem cells, become committed granulocyte-monocyte stem cells, and differentiate to myeloblasts, promyelocytes, myelocytes, metamyelocytes, and segmented neutrophils (Robert, et al., Principles and Practice of Hematology, Lipincott, Philadelphia, 1995). Hematologic diseases such as myeloid leukemia are associated with the deregulation of myeloid differentiation (Velculescu, et al., Science, 270, 484-487, 1995). Thus, the identification and analysis of genes abnormally expressed myeloid leukemia cells is an important approach towards understanding the onset of the leukemic state in these cell types.

By using SAGE and GLGI, an unbiased genome-wide analysis was performed to identify the genes abnormally expressed in leukemic cells with t(9;11). The abnormal expression in leukemic cells is defined as a change in expression relative to control cells, such as wild-type cells. Any detectable change in expression will suffice to identify abnormal expression, such as a two-fold or a three-fold change in expression. Preferably, the change in expression is at least a five-fold change in expression. After analyzing the data, 244 SAGE tags were identified that differed in gene expression level by more than 5 fold between three leukemia samples and a normal CD15+ library. The 244 SAGE tags were converted to 223 genes or ESTs or novel 3′ ESTs with GLGI.

To solve issues related to determining the correct sequence corresponding to a SAGE tag among all of the matched sequences, the GLGI technique is performed to convert SAGE tags to the 3′ end of their corresponding cDNA. By using GLGI, the sequences of the novel tags were identified; GLGI also further clarified that multiple-matched tags were discovered. Currently, 223 of 244 tags were converted to the corresponding genes, or lengthier ESTs, with the following alterations in expression detected: 118 genes were down-regulated and 29 genes were up-regulated in leukemia samples by at least 5 fold. Also, 64 genes were turned off and 12 genes were turned on in leukemia samples and 21 tags were unconfirmed. Among these genes/ESTs, 155 matched to known genes/ESTs, and 68 are novel 3′ ESTs without matches in the current database.

A. Preparation of Myeloid Progenitor Cells

In one study, 244 genes that are abnormally expressed in myeloid progenitor cells with an MLL-AF9 fusion were identified. As used herein, the term “progenitor cell” refers to a hematopoietic cell having the CD15⁺ cell surface antigen (stem cells and colony-forming units). Such cells may be isolated using techniques well known to those of skill in the art.

Human bone-marrow mononuclear cells were obtained from Poietics™ (East Rutherford, N.J.). Poietics™ offers human stem cells derived from the hematopoietic system. Bone marrow cells are obtained through an IRB-approved donor program operated by BioWhittaker. Cells are offered in several grades of purity from unprocessed marrow to cells selected by both positive and negative immunoselection. Poietics™ provides the cells as either fresh or cryopreserved cells.

The suspensions of mononuclear cells can be enriched for stem/progenitor cells by positive selection of CD15⁺ cells using magnetic beads (Dynal, Oslo, Norway). CD15⁺ beads (Dyna Beads M-450) are first applied into mononuclear cells and rotated for 20 minutes. The bead/cell complexes are then isolated using a magnetic tube holder. After the washing step, the CD15⁺ cells can be released from the beads by adding 50 U/ml of Chymodiactin™ (Bootes Pharmaceutical, Lincolnshire, Ill.) in RPMI 1640 (Sigma Chem. Co., St. Louis, Mo.) and incubating for 15 minutes at 37° C. The cells released from the beads may then be evaluated for CD15⁺ purity by staining with CD15⁺ FITC/CD117 PE monoclonal antibody to CD15⁺ (BD Oncomark, BD Sciences, San Jose, Calif.). To stain the cells, the anti-CD15⁺ monoclonal antibody was added to the cells and incubated for 15 minutes on ice, followed by quantitation of stained cells using a FACScan™ flow cytometer (Becton Dickenson, San Jose, Calif.; Smith et al., Experimental Hematology 21, 870-877, 1993).

B. Serial Analysis of Gene Expression (SAGE)

SAGE was employed to identify 244 genes that are abnormally expressed in CD15⁺ myeloid progenitor cells with an MLL-AF9 fusion. SAGE (Serial Analysis of Gene Expression) (Velculescu et al., Science, 270, 484-487, 1995) is a powerful technique that can be used as a tool for the analysis of gene expression. This technique was used in order to obtain the broadest identification of expressed genes and to provide quantitative information for each identified gene.

SAGE is a technique for rapidly obtaining qualitative and quantitative surveys of cellular gene expression that involves two conceptual steps. First, short nucleotide sequence tags (e.g., 9 to 10 base pairs (bp)), typically containing enough information to uniquely identify a transcript, are generated from cDNA using restriction endonucleases that cleave at known recognition sites, herein referred to as anchor sites. Second, concatenation of short sequence tags by ligation, producing a single clonable nucleic acid fragment, facilitates the efficient analysis of transcripts by serially sequencing the multiple tags constituting the single nucleic acid fragment. Typically, the fragment is ligated into a vector (i.e., cloned) for ease of manipulation, including sequence determination. Boundaries between tags are preserved to enable the separate qualitative and quantitative characterization of individual gene expression, as will become apparent from the description of SAGE provided below.

Double-stranded cDNA is synthesized from mRNA using a biotinylated oligo(dT) primer. In an exemplary embodiment, the cDNA is synthesized as described in Lee et al., Proc. Natl. Acad. Sci. (USA), 98, 3340-3345, 2001). Briefly, the DNA from the CD15⁺ cells is extracted for use in the SAGE/GLGI techniques described below. The CD15⁺ cells are lysed directly with TRIZOL reagent (Life Technologies, Rockville, Md.) for isolation of total RNA according to the manufacturer's instructions. The mRNA is purified from 5 μg of total RNA with oligo(dT)25 beads (Dynal) by following the manufacturer's protocol. This mRNA preparation then serves as a template for cDNA synthesis using a cDNA synthesis kit (Life Technologies) according to the manufacturer's instructions, but with the following exceptions. First, to prevent the inclusion of poly(dA/dT) sequences in the cDNA templates, 5′-biotinylated and 3′-anchored oligo(dT) primers were used for reverse transcription. Second, to increase the cDNA yield, the first-strand synthesis reaction was repeated three times under the following conditions: the primary reaction was incubated at 37° C. for 30 minutes, heated at 65° C. for 2 minutes, and incubated again at 37° C. with the addition of 2 μl of Maloney murine leukemia virus reverse transcriptase.

The cDNA is then cleaved with a restriction endonuclease, which is used in the SAGE method as an anchoring enzyme. A suitable anchoring enzyme is expected to cleave most transcripts at least once. Typically, restriction endonucleases with 4-bp recognition sites are used for this purpose because they cleave every 256 bp (4⁴) on average, whereas most transcripts are considerably longer than 256 bases. However, enzymes recognizing longer sites may also be used. For example, enzymes having recognition sites that are partially or fully degenerate, such as the recognition site (5′-A′CPuPyGT-3′, where Pu=purines A or G, Py=pyrimidines C, T, or U, and ′″″ denotes the cleavage site) for AflIII, may be used in SAGE. Additionally, enzymes having longer recognition sites may be used to cleave nucleic acid that has been modified (e.g., methylated) to alter the cleavage frequency. Further, enzymes having sites that happen to occur more frequently in a given genome than would be expected based solely on statistical calculations (e.g., because of non-random GC content or a genomic bias with respect to a partial or full recognition site sequence in a genome) may also be used in SAGE. Analogously, enzymes having restriction endonuclease activities that are affected by environmental conditions, such as the ionic strength of buffers (e.g., “star” activity) are also contemplated for use in SAGE. Although anchoring enzymes that cleave to produce blunt ends in double-stranded DNA may be used, it is preferred that the anchoring enzymes cleave duplex DNA to produce cohesive termini. The property common to all suitable anchor enzymes is their capacity to cleave the cDNA corresponding to most, and preferably all, mRNAs in a given cell or cell type, and to cleave at a known site. Exemplary anchoring enzymes are type II restriction endonucleases that include, but are not limited to, AciI, AluI, BfaI, BssKI, BstUI, Csp6I, DdeI, DpnI, DpnII, Fnu4HI, HaeIII, HhaI, HinfI, HinP1I, HpaII, MboI, MseI, MspI, NlaIII, RsaI, Sau3AI, TaiI, TaqI, and Tsp509I.

Following cleavage of the cDNA with the anchor enzyme, the 3′ portion of each cleaved cDNA was then isolated by binding the 3′ biotin to streptavidin beads. This process provides a unique site on each transcript that corresponds to the restriction site located closest to the polyadenylate [poly(A)] tail. This cDNA preparation was then divided into two approximately equal cDNA sample portions and each sample was ligated via the anchor site to one of two linkers containing a cleaved anchor site and a type IIS restriction site (tagging system). Type IIS restriction endonucleases cleave at a defined distance up to 20 bp away from their asymmetric recognition sites (Szybalski et al., Gene, 40, 169, 1985). The linkers are designed so that cleavage of the ligation products with the tagging enzyme results in release of the linker with a short piece of the cDNA. A typical type IIS restriction enzyme is BsmFI. Other exemplary Type IIS restriction enzymes that may be used in this technique include, but are not limited to, AlwI, BbsI, BbvI, BpmI, BsaI, BsgI, BsmI, BsmBI, BsmFI, BspMI, BsrI, BsrDI, EarI, Eco57I, FokI, HgaI, HphI, MboII, MnlI, PleI, SapI, and SfaNI.

After blunt ends are created, the two pools of released tags are ligated to each other. Ligated tags (ditags) then serve as templates for polymerase chain reaction (PCR) amplification with primers specific to each linker. The resulting amplification products contain two tags linked tail to tail, flanked by sites for the anchoring enzyme. In the final sequencing template, this results in 4 bp of punctuation per ditag. The analysis of ditags, formed before any amplification steps, allows the elimination of potential distortions introduced by PCR. Because the probability of any two tags being coupled in the same ditag is small, even for abundant transcripts, repeated ditags potentially produced by biased PCR can be excluded from analysis without substantially altering the final results. Cleavage of the PCR product with the anchoring enzyme allows isolation of the ditags, which can then be concatenated by ligation, cloned, and sequenced.

In an exemplary SAGE analysis employed in the invention, the double-stranded cDNA was produced as described above and then cleaved with NlaIII as the anchoring enzyme. The 3′ restriction fragments were isolated by binding to magnetic streptavidin beads (Dynal). The bound cDNA was then subdivided into two pools (A and B) and the DNA in each pool was ligated to linker A (pool A) or linker B (pool B). The pools were then extensively washed to remove unligated linkers. The linkers and adjacent tag were released by cleavage with BsmFI as the tagging enzyme. The resultant overhangs were blunted by a fill-in reaction using Pfu polymerase (Stratagene) and the pools were combined and ligated to each other. The ligation product was then amplified using PCR. The products generated by PCR were then analyzed using polyacrylamide gel electrophoresis and fractionated products were excised from the gel. The PCR products were then cleaved with the restriction endonuclease initially used as the anchoring enzyme and the band containing the ditags was excised and self-ligated. This ligation generates concatenated ditags, which can then be separated by PAGE, the desired products excised, and the excised products cloned into a plasmid for further analysis. For example, the products are cloned into pZero vector (Invitrogen). Positive clones are screened by using PCR with M13 reverse and M13 forward primers recognizing sequences located in the vector. Sequencing reactions were performed with a PE big-dye kit (PE Applied Biosystems, Foster City, Calif.) using the M13 reverse primer, following the manufacturer's instructions.

In certain embodiments using a modified SAGE, the BsmFI-released fragments containing the SAGE tags were gel purified before being used for ditag formation to provide high-quality tags for SAGE analysis. SAGE-tag sequences were identified using the Big-Dye sequencing kit and an ABI377 sequencer (Perkin-Elmer Applied Biosystems). Tag sequences were identified with SAGE 300 software.

C. Generation of Longer cDNA Fragments from SAGE for Gene Identification (GLGI).

Some problems may attend efforts to use SAGE tag sequence for gene identification. First, many SAGE tags identified have no match to known sequences in databases (Zhang et al., Science 276, 1268-1272, 1997; Velculescu et al., Cell 88, 243-251, 1997). These tags may represent previously unidentified genes; nevertheless, it is difficult to use the tag information for further characterization of the corresponding genes. Second, the converse problem exists in that certain SAGE tag sequences have multiple matches with sequences in the databases. Often, sequences containing identical SAGE tag sequences have no similarity to each other and no other discernable relationship with each other except for sharing the same SAGE tag sequence. This feature makes it difficult to determine the correct sequence in a particular tissue.

A technique called the generation of longer cDNA fragments from SAGE tags for gene identification (GLGI) has been developed to overcome these problems (Chen et al., Proc. Natl. Acad. Sci. (USA), 97, 349-353, 2000). Key features of this technique include the use of an oligonucleotide containing a SAGE tag sequence as a sense primer, and an anchored oligo(dT) as the antisense primer, for PCR amplification catalyzed by, e.g., platinum Taq DNA polymerase (Invitrogen). By using this approach, a SAGE tag sequence can be converted into a longer cDNA fragment containing up to several hundred bases from the SAGE tag to the 3′ end of the corresponding cDNA. The development of the GLGI technique overcomes the two obstacles discussed above and has wide application in SAGE-related techniques for global analyses of gene expression.

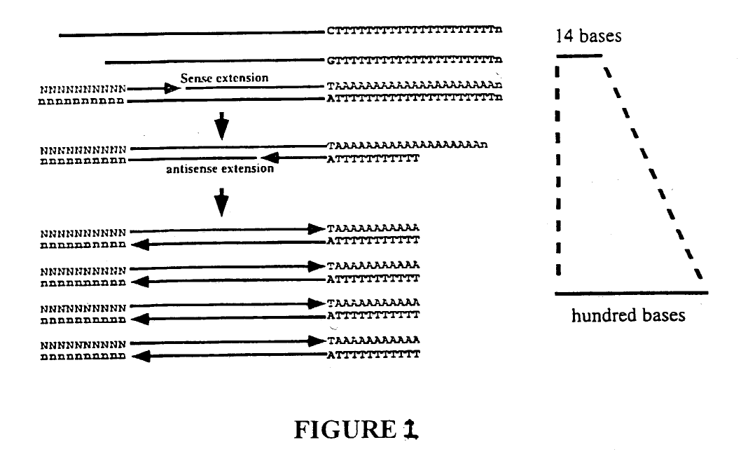

FIG. 1 schematically illustrates the GLGI process. In this process, a first-strand cDNA synthesized by extending an oligo(dT) primer is used for PCR. In the first PCR cycle, the template with the SAGE tag binding site is annealed to the sense primer and extended to the end of the template. In the second PCR cycle, extension occurs only from an anchored oligo(dT) primer annealed to the 5′ ends of poly(dA) tails, thereby avoiding PCR products of heterogeneous lengths due to variation in primer binding locations within long poly(dA) tracts. Exponential amplification occurs only for the template with the SAGE tag binding site. The result of GLGI is an increase in gene-specific sequence from the approximately 10 bases provided by a SAGE tag up to the entire sequence of a gene from the anchor enzyme recognition site at the 3′ end of the transcribed DNA to the DNA encoding the poly(dA) tail.

Elaborating on the technique further, each SAGE tag typically contains only a 10-base sequence. To increase the length of the primers for efficient PCR priming, 5′-CATG-3′, a NlaIII recognition site used for collecting SAGE tag fragments (Velculescu et al., Science, 270, 484-487, 1995), is added immediately 5′ to the SAGE tag. A BamHI recognition site, 5′-GGATCC-3′, is added 5′ to these sites to increase the primer size and to provide a potential site for subcloning. For the anchored oligo(dT) primers, a single-base anchor, dA, dG, or dC, is attached to the 3′ end of the oligo(dT) primer (Khan et al., Nucleic Acids Res., 19:1715, 1991; Kiriangkum et al., Nucleic Acids Res., 20:3793-94, 1992; Liang et al., Science, 257, 967-970, 1992; Liang et al., Nucleic Acids Res., 22, 5763-5764, 1994; Wang et al., Proc. Natl. Acad. Sci. (USA), 95, 11909-11914, 1998). Recent studies have shown that the preferred length for an oligo(dT) sequence in this technique is dT11; however, it should be understood that different numbers of dT nucleotides ranging from 8 to 20, or longer, may be used.

The GLGI technique permits amplification of a particular template corresponding to a particular SAGE tag by using a combination of a typically gene-specific sense primer containing a SAGE tag sequence and an anchored oligo(dT) primer (FIG. 1). In this process, only cDNA containing the binding sequences for the SAGE tag will be functional templates in the first PCR cycle. In the second cycle, extension will occur only from templates that allow the anchored oligo(dT) primer to anneal at the 5′ end of a poly(dA) sequence with the anchored nucleotide correctly paired to the nucleotide immediately 5′ of the poly(dA) sequence. Extension of all other anchored primers annealed along the poly(dA) sequences will be blocked because of the presence of the unbound anchor nucleotide. Extension of these templates will eliminate unnecessary poly(dA)/(dT) sequences. Only cDNA templates containing the SAGE tag sequence will undergo exponential amplification in the following PCR cycles, thereby ensuring that PCR amplification will produce nucleic acid copies of the same size.

In an exemplary PCR protocol used in GLGI, Pfu DNA polymerase (Stratagene) is used with 10×buffer (200 mM Tris-HCl, pH 8.4/500 mM KCl). MgCl₂ (1.5 mM) was added in each reaction to increase the [Mg²⁺]. The PCR mixture contained 1×buffer, 1.5 mM MgCl₂, 0.3 mM dNTP, 0.04 units/μl platinum Taq polymerase, 3 ng/μl sense primer, and 1.5 ng/μl anchored oligo(dT) primer (single or mixture) in a final volume of 20 or 50 μl. The PCR reactions were performed first at 94° C. for 1 minute, followed by five cycles at 94° C. for 20 seconds, 50-53° C. for 20 seconds, and 72° C. for 20 seconds. The conditions were then changed to 25 cycles at 94° C. for 20 seconds, 60° C. for 20 seconds, and 72° C. for 20 seconds. The reactions were kept at 72° C. for 5 minutes for the last cycle.

To amplify longer sequences from SAGE tags, PCR is performed with each sense primer containing the SAGE tag sequence and individual or mixed anchored oligo(dT) primers, combined with cDNAs from the desired cells generated by oligo(dT) priming. The PCR products are subjected to electrophoresis through an agarose gel and cloned into a vector for sequence analysis. The reaction can be simplified into a single reaction by using a combination of dA-, dG-, and dC-anchored oligo(dT) primers.

As indicated above, when matching SAGE tag sequences in databases, a single SAGE tag may match several sequences and, as such, a SAGE tag itself may not be sufficient to serve as a unique identifier for a particular sequence. It is important to distinguish which one of the matched sequences is the correct sequence corresponding to the particular SAGE tag, and this ability is achieved using GLGI. To avoid the uncertainty when different sequences are expressed from different tissues, it will be preferable to generate the fragment from the same tissue used to generate the SAGE tag.

D. Nucleic Acids of the Invention and Methods of Achieving Their Recombinant Expression in Cells

Recombinantly expressed proteins may be purified according to known methods, such as precipitation (e.g., ammonium sulfate), HPLC, FPLC, ion exchange chromatography, affinity chromatography (including immunoaffinity chromatography) or via various size separation techniques (sedimentation, gel electrophoresis, gel filtration).

Any reference to a nucleic acid of the invention should be understood as encompassing a vector comprising that polynucleotide and a host cell containing that vector or nucleic acid and, in some cases, capable of expressing the protein product of that nucleic acid. Cells expressing nucleic acids of the invention will be useful in certain therapeutic implications, and methods of making and using such cells are described below.

The nucleic acid sequences disclosed in SEQ ID NOS: 1-244 include genomic DNA, cDNA, mRNA, as well as recombinant and synthetic sequences and partially synthetic sequences, which may encode an entire protein, polypeptide, or allelic variant thereof.

Nucleic acids having sequences corresponding to any one of SEQ ID NOS: 1-244 may be obtained from genomic DNA, i.e., cloned directly from the CD15⁺ myeloid progenitor cells. However, the nucleic acid also could be obtained from complementary DNA (cDNA). Also contemplated is a cDNA plus a natural intron or an intron derived from another gene; such engineered molecules are sometime referred to as “mini-genes.” These and other nucleic acids of the invention are useful as markers of hematopoietic cell gene expression, and therefore are markers for hematopoietic cells generally and hematopoietic cells exhibiting a particular physiology more particularly.

The term “cDNA” is intended to refer to DNA prepared using messenger RNA (mRNA) as a template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are targets in antisense methods of modulating gene expression.

It may be advantageous to combine portions of genomic DNA with cDNA or synthetic sequences to generate specific constructs. For example, where an intron is desired in the ultimate construct, a genomic clone is suitable. The cDNA or a synthesized polynucleotide may provide more convenient restriction sites for the remaining portion of the construct and, therefore, would be used for the rest of the sequence.

As used in this application, the term “a nucleic acid encoding a protein from CD15⁺ myeloid progenitor cells” refers to a nucleic acid molecule that has been isolated from total cellular nucleic acid of a CD15⁺ myeloid progenitor cell. In preferred embodiments, the nucleic acid molecule has a sequence essentially as set forth in any one of SEQ ID NOS: 1-244. The term “functionally equivalent codon” is used herein to refer to codons that encode the same amino acid (see Table 4), such as the six codons for arginine or serine, and also refers to codons that encode biologically equivalent amino acids, as discussed in the following pages.

TABLE 4

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The invention also encompasses DNA segments that are complementary, or essentially complementary, to the sequences set forth in any one of sequences of SEQ ID NO: 1 to SEQ ID NO: 244. Nucleic acid sequences that are “complementary” are those that are capable of base-pairing according to the standard Watson-Crick complementarity rules. That is, gaunidylate (deoxyguanidylate) pairs with cytidylate (deoxycytidylate) and adenylate pairs with uridylate (thymidylate). Inclusion of less common bases such as inosine, 5-methylcytosine, 6-methyladenine, hypoxanthine and others in hybridizing sequences does not interfere with pairing, and is contemplated as falling within the scope of the invention.

As used herein, the term “complementary sequences” means nucleic acid sequences that are substantially complementary, as may be assessed by the same nucleotide comparison set forth above, or as defined as being capable of hybridizing to a nucleic acid having a sequence of SEQ ID NOS: 1 to 244 under stringent conditions such as those described herein. Those of skill in the art will understand what is meant by stringent conditions and are referred to page 11.45 of Molecular Cloning: A laboratory Manual, 2^{nd} Ed. Cold Spring Harbor Laboratory Press, Cold Spring Harbor, N.Y., or the conditions set forth in the Summary of the Invention, above.

The term “nucleic acid” as used herein also includes antisense nucleic acids. Antisense nucleic acids are able to bind to the specific mRNA through base-pairing and then, e.g., by degradation of the mRNA, interfere with protein expression.

Alternatively, the hybridizing nucleic acids may be shorter (i.e., oligonucleotides). Sequences of about 17 bases long should occur only once in the human genome and, therefore, should suffice to specify a unique target sequence. Nucleotide sequences of this size that specifically hybridize to any of the nucleic acids sequences in Tables 2-4 are useful as probes or primers. As used herein, an oligonucleotide that “specifically hybridizes” to a nucleic acid in Tables 2-4 means that hybridization under suitably (e.g., high) stringent conditions allows discrimination of one or a few hybridizing sequences, preferably one sequence, from other genes. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

Suitable hybridization conditions are well known to those of skill in the art. In certain applications, it is appreciated that lower stringency conditions may be required. Under these conditions, hybridization may occur even though the sequences of the interacting strands are not perfectly complementary, being mismatched at one or more positions. Conditions may be rendered less stringent by, e.g., increasing salt concentration and/or decreasing temperature.

One method of using probes and primers of the invention is in the search for gene expression in human cells. Normally, the target DNA will be a genomic or cDNA library, although screening may involve analysis of RNA molecules. By varying the stringency of hybridization, and the region of the probe (i.e., the sequence of the probe, corresponding to a subset of one of the sequences set forth at SEQ ID NOS: 1-244), different degrees of homology may result in hybridization.

Given the above disclosure of the nucleic acid constructs, it is possible to produce the gene product of any of the genes corresponding to SEQ ID NO: 1 through SEQ ID NO: 244 by routine recombinant DNA/RNA techniques. A variety of expression vector/host systems may be utilized to contain and express the coding sequence. These include, but are not limited to, microorganisms such as bacteria transformed with recombinant bacteriophage, plasmid, phagemid, or cosmid DNA expression vectors; yeast transformed with yeast expression vectors or shuttle vectors; insect cell systems infected with viral expression vectors (e.g., baculovirus); plant cell systems transfected with virus expression vectors (e.g., Cauliflower Mosaic Virus, CaMV; Tobacco Mosaic Virus, TMV) or transformed with bacterial expression. vectors (e.g., Ti or pBR322 plasmid); or even animal cell systems. Mammalian cells that are useful in recombinant protein productions include, but are not limited to, VERO cells, HeLa cells, Chinese hamster ovary (CHO) cells, COS cells (such as COS-7), WI38, BHK, HepG2, 3T3, RIN, MDCK, A549, PC12, K562 and HEK 293 cells.

In other embodiments, expression vectors may be used to introduce the genes of the invention into host cells to produce recombinant cells. The invention contemplates expression via cassettes, which requires that appropriate signals or various regulatory elements to be provided in the vectors, such as enhancers, promoters, expression factor binding sites, and terminators. These signals and/or elements collectively control expression of the genes of interest in the host cells of interest.

Throughout this application, the term “expression construct” or “expression vector” is meant to include any type of genetic construct containing a nucleic acid coding for a gene product in which part or all of the nucleic acid encoding sequence is capable of being transcribed. The transcript may be translated into a protein and this process may be facilitated by inclusion of a ribosome binding site and/or a stop codon(s) in the expression vector, but it need not be. In certain embodiments, expression includes both transcription of a gene and translation of the cognate mRNA into a protein gene product.

The nucleic acid encoding a gene product is under transcriptional control of a promoter. A “promoter” refers to a DNA sequence recognized by the native synthetic machinery of the cell, or introduced synthetic machinery, required to initiate the specific transcription of a gene. The phrase “under transcriptional control” means that the promoter is in the correct location and orientation in relation to the coding region of interest to control RNA polymerase initiation and appropriate extension of the nascent mRNA corresponding to the gene.

The term promoter will be used herein to refer to at least one member of a group of transcriptional control modules that are clustered around the initiation site for RNA polymerase II. Additional promoter elements regulate the frequency of transcriptional initiation. Typically, these are located in the region 30-110 bp upstream of the transcription, start site, although a number of promoters have recently been shown to contain functional elements downstream of the start site as well, and constructs containing such promoters are contemplated by the invention. The spacing between promoter elements frequently is flexible, so that promoter function is preserved when elements are inverted or moved relative to one another. In the tk promoter, for example, the spacing between promoter elements can be increased to 50 bp before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the cell of interest. Thus, where a bacterial host cell is used, it is preferable to position the nucleic acid coding region adjacent to, and under the control of, a promoter that is capable of being expressed in a bacterial cell. Generally speaking, such a promoter is a bacterial or a phage promoter.

Suitable promoters for prokaryotes include, for example, the trp promoter (inducible by tryptophan deprivation), the lac promoter (inducible with the galactose analog IPTG), the β-lactamase promoter, and the lambda phage-derived P_{L} promoter (derepressible by temperature variation if the cIₜₛ marker is also used in the expression system). Other useful promoters include those for alpha-amylase, protease, Spo2, spac, and tac promoters. Especially preferred promoters include the kanamycin resistance promoter, G13, and the endogenous or native promoter for whichever gene is being introduced, e.g., the 9-α-hydroxylase promoter.

Promoters that may be used for expression in yeast include the 3-phospho-glycerate kinase promoter and those for other glycolytic enzymes, as well as promoters for alcohol dehydrogenase and yeast phosphatase. Also suited are the promoters for transcription elongation factor (TEF) and lactase. Mammalian expression systems generally may include the SV40 promoter, known constitutive promoters functional in such cells, or regulatable promoters, such as the metallothionein promoter, which is controlled by heavy metals or gluco-corticoid concentration.

All of the above promoters, well known and readily available to those of skill in the art, can be used to obtain high-level expression of the coding sequence of interest. The use of other viral or mammalian cellular, viral or bacteriophage promoters, which are well-known in the art, to achieve expression of a coding sequence of interest are contemplated as well, provided that the levels of expression are sufficient for a given purpose. By employing a promoter with well known properties, the level and pattern of expression of the protein of interest following transfection or transformation can be optimized.

Selection of a promoter that is regulated in response to a specific physiologic or synthetic signal(s) can permit inducible or derepressible (i.e., controllable) expression of the gene product. Several such promoter systems are available for production of viral vectors. One exemplary system is the ecdysone system (Invitrogen, Carlsbad, Calif.), which is designed to allow regulated expression of a gene of interest in mammalian cells. It consists of a tightly regulated expression mechanism that allows barely detectable basal level expression of a transgene, but over 200-fold inducibility of expression

Translation control sequences include a ribosome binding site (RBS) in prokaryotic systems, whereas in eukaryotic systems translation may be controlled by a “TATA” box sequence which may also contain an initiation codon such as AUG (ATG). Another regulatory element contemplated for use in the invention is an enhancer. These are genetic elements that increase, or enhance, transcription; enhancers may be located a considerable distance from a functionally related coding region (separation of several Kb or more), the relative locations of enhancer and coding region is not specific (the enhancer may be 5′, 3′ or internal to the coding region), and the orientation of the enhancer itself is not specific (some enhancers function in inverted orientation). Promoters and enhancers are often overlapping and contiguous, often seeming to have a very similar modular organization. Enhancers useful in the invention are known in the art and will depend on the particular expression system being employed (Scharf et al Results Probl Cell Differ, 20, 125-62, 1994; Bittner et al, Methods in Enzyrnol, 15, 516-544, 1987).

There are a number of ways in which expression vectors may be introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genomes and express viral genes stably and efficiently have made them attractive candidates for the transfer of foreign genes into mammalian cells (Ridgeway, In: Rodriguez R L, Denhardt D T, eds. Vectors: A survey of molecular cloning vectors and their uses. Stoneham: Butterworth, pp. 467-492, 1988; Nicolas et al., In: Vectors: A survey of molecular cloning vectors and their uses, Rodriguez & Denhardt (eds.), Stoneham: Butterworth, pp. 493-513, 1988; Baichwal et al., In: Gene Transfer, Kucherlapati ed., New York, Plenum Press, pp. 117-148, 1986; Temin, In: gene Transfer, Kucherlapati (ed.), New York: Plenum Press, pp. 149-188, 1986).

Several non-viral methods for the transfer of expression constructs into cultured bacterial cells are contemplated by the invention. This section provides a discussion of methods and compositions of non-viral gene transfer. DNA constructs of the invention are generally delivered to a cell and, in certain situations, the nucleic acid or the protein to be transferred may be transferred using non-viral methods. The non-viral methods include calcium phosphate precipitation (Graham et al., Virology, 52:456-467, 1973; Chen et al., Mol. Cell. Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau et al., Biochim. Biophys. Acta, 721:185-190, 1982; Fraley et al., Proc. Natl. Acad. Sci. (USA), 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. (USA), 84:8463-8467, 1987), gene bombardment using high velocity microprojectiles (Yang et al., Proc. Natl. Acad. Sci USA, 87:9568-9572, 1990), conjugation (Gavigan et al. In: Tanya Parish and Neil G. Stoker (eds). Mycobacteria Protocols, pp. 119-128 1998. Humana Press, Totowa. N.J.) and receptor-mediated transfection (Wu et al., J. Biol. Chem., 262:4429-4432, 1987; Wu et al., Biochemistry, 27:887-892, 1988; Wu et al., Adv. Drug Delivery Rev., 12:159-167, 1993).

The expression construct also may be entrapped in a liposome. Liposomes are vesicular structures characterized by a phospholipid bilayer membrane and an inner aqueous medium. Multilamellar liposomes have multiple lipid layers separated by an aqueous medium. They form spontaneously when phospholipids are suspended in an excess of aqueous solution. The lipid components undergo self-rearrangement before the formation of closed structures entrap water and dissolved solutes between the lipid bilayers (Ghosh et al., In: Liver diseases, targeted diagnosis and therapy using specific receptors and ligands, Wu et al. ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vehicles for use in gene delivery.

Also contemplated in the invention are various commercial approaches involving “lipofection” technology. In certain embodiments of the invention, the liposome may be complexed with a hemagglutinating virus (HVJ). This has been shown to facilitate fusion with the cell membrane and to promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J Biol. Chem., 266:3361-3364, 1991).

Other vector delivery systems which can be employed to deliver a nucleic acid encoding a given gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu et al., 1993, supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu et al., 1987, supra) and transferrin (Wagner et al., Proc. Natl. Acad Sci. (USA), 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci. (USA) 91:4086-4090, 1994) and epidermal growth factor (EGF) has also been used to deliver genes to squamous carcinoma cells (Myers, EPO 0273085).

Another embodiment of the invention for transferring a naked DNA expression construct into cells may involve particle bombardment. This method depends on the ability to accelerate DNA coated microprojectiles to a high velocity, allowing them to pierce cell membranes and enter cells without killing them (Klein et al., Nature, 327:70-73, 1987). Several devices for accelerating small particles have been developed. One such device relies on a high-voltage discharge to generate an electrical current, which in turn provides the motive force (Yang et al., Proc. Natl. Acad. Sci. (USA), 87:9568-9572, 1990). The microprojectiles used to date have consisted of biologically inert substances such as tungsten or gold beads.

E. Identification of Factors Expressed by the Genes

It is contemplated that one or more of the polynucleotides disclosed herein (SEQ ID NOS: 1-244) will encode a colony stimulating factor (CSF) akin to other human CSFs (e.g., GM-CSF, G-CSF, eosinophilic CSF, erythroid potentiating activity) (Nicola, et al., Blood 54:614-627, 1979; Golde et al., Proc. Natl. Acad. Sci. (USA) 77, 593-596, 1980; Lusis, Blood 57, 13-21, 1981; Abboud et al., Blood 58, 1148-1154, 1981; Okabe, J. Cell. Phys. 110, 43-49, 1982; Fauser et al., Stem Cells, 1, 73-80, 1981).

Those of skill in the art are aware of functional assays that may be performed to test for CSF activity. It is contemplated that the CSF biological properties that one or more of the gene products of the invention will exhibit include differentiation of progenitor cells to all major blood types as well as differentiation of leukemic cells. The following section provides brief descriptions of assays for determining CSF activity. Such assays may be used to test the activities of the gene products of the invention.

1. Assay for GM-CSF Activity

GM-CSF activity is generally tested on human bone marrow (BM) cells cultured with serial dilutions of test samples (of the gene products) in semi-solid agar. BM from healthy human volunteers is appropriately diluted, e.g., 1:5 in a buffer such as a phosphate-buffered saline (PBS), and separated by density gradient centrifugation on Ficoll-Hypaque. Approximately 10⁵ separated cells are plated in 1 ml of 0.3% agar culture medium that includes supplemented McCoy's 5 A medium and 10% heat-inactivated fetal calf serum (FCS; Broxmeyer, et al., Exp. Hematol., 5, 87-102, 1977). Serial dilutions of a laboratory standard or test samples (10%; v/v) in RPMI 1640 with 10% FCS are added to the above mixture as a standard. Cultures are scored for colonies (defined as greater than 40 cells/aggregate) and morphology is assessed after 7 and 14 days of incubation. GM-CSF units are determined from dose response curves and expressed as U/ml, where 50 U is the CSF concentration stimulating half-maximal colony number to develop (Nicola et al., J. Biol. Chem., 258, 9017-9021, 1983).

2. Assay for Colony Stimulating Factor for BFU-E and CFU-GEMM

A colony assay for human BFU-E and CFU-GEMM may be performed according to a method previously described by Li Lu, et al. Blood 61, 250-256, 1983. Human bone marrow cells are subjected to a density cut with Ficoll-Hypaque (density 1.077 gm/cm³; Pharmacia Fine Chemicals, Piscataway, N.J.) and the low density cells are suspended in RPMI 1640 containing 10% FCS at 2×10⁷ cells/ml and placed for adherence on Falcon tissue cultures dishes (#3003, Becton Dickinson and Co., Cockeysville, Md.) for 1.5 hours at 37° C. The nonadherent cells are depleted of T lymphocytes by resetting with neuraminidase-treated sheep erythrocytes. Medium conditioned by leukocytes from patients with hemochromatosis in the presence of 1% (v/v) phytohemagglutinin (PHA) (Li Lu, et al., 1983, supra) as positive control or serial dilutions of test samples is then added at 5% (v/v) to 5×10⁴ of these low-density, non-adherent and T lymphocyte-depleted bone marrow cells in a 1 ml mixture of Iscove's modified Dulbecco medium (GIBCO, Grand Island, N.Y.), 0.8% methylcellulose, 30% FCS, 5×10⁻⁵ M 2-mercaptoethanol, 0.2 mM Hemin, and one unit of erythropoietin (Hyclone, or Connaught Labs., Willowdale, Ontario, Canada). The addition of Hemin is necessary to obtain optimal cloning efficiency (Li Lu et al., Exp. Hematol., 11, 721-729, 1983). Dishes are incubated in a humidified atmosphere of 5% CO₂ in air at 37° C. After 14 days of incubation, colonies are scored and morphology assessed.

3. Differentiation of Leukemic Cell Lines

Differentiation of leukemic cells lines in vitro can be achieved by a variety of nonphysiologic (e.g., DMSO, phorbol diesters) and physiologic (e.g., retinoic acid, vitamin D₃) inducers (Koeffler et al., Blood, 62, 709 1983). Murine G-CSF is known to be a potent inducer of differentiation of WEHI-3B (D+) murine myelomonocytic leukemia cells, whereas Interleukin 3 lacks this activity (Nicola et al. Immunol. Today, 5, 76, 1984).

A CSF of the invention can be tested for leukemia differentiating activity (GM-DF) in a clonal assay system (Metcalf, Int. J. Cancer, 25, 225, 1980; Fibach, et al., J. Cell. Physiol., supra) using murine WEHI-3B (D+) and human HL-60 promyelocytic leukemia cell lines. Quantitation of GM-DF is obtained by incubation of leukemic cells in agar with serial dilutions of the CSF.

4. Morphology and Cytochemistry

Morphological and cytochemical analyses of colonies are performed using alpha-naphthylacetate esterase (ANAE) and luxol fast blue (LFB) stains, as markers of the monocyte, macrophage and eosinophil granulocyte lineages respectively (Platzer et al., Immunobiology, 172(3-5), 185-93 1986; Platzer, et al., J Exp. Med., 162(6), 1788-801 1985; Platzer et al., Blood Cells, 14(2-3), 463-9 1988; Platzer et al., Blut., 54(3), 129-36, 1987). If the presence of a CSF produces an increase in the number of colonies containing polymorphonuclear cells (by hematoxylin stain), the number of LFB⁺ cells, and the intensity of ANAE stain, then the CSF induces differentiation along the macrophage as well as granulocyte pathways.

In other assessments of the lineage specificity of colonies, intact agar cultures is dehydrated on glass slides and stained for acetylcholinesterase. A counterstain that is utilized for nuclei is methyl green. The colonies are then examined microscopically; the nuclear configuration, cell shape, and cell size allow differentiation between the cell lineages, such as granulocytic, mononuclear, fibrocytic stromal and megakaryocytic. In addition, the megakaryocyte cytoplasm stains brown due to the presence of acetylcholinesterase. Clusters of three or more megakaryocytes may be taken as representing a megakaryocyte colony.

5. Preparation and Purification of Factors from Conditioned Media of Cell Lines

It is possible to screen a library of cell lines to identify a cell line that contains a gene of the invention that encodes a CSF of interest. Like the identification of a cell line containing such a gene, it is a matter of routine skill in the art to grow the cell line in culture and purify quantities of the CSF from the conditioned media of the cell culture. For purification of CSF activity from conditioned medium, confluent cell cultures are intermittently cultured in medium containing 0.2% FCS. After 48-72 hours, the conditioned medium is harvested, cells and cell debris removed by centrifugation (20 min, 10,000×g), and stored at −20° C. until use.

The proteins from the conditioned medium can then be purified by precipitation with ammonium sulfate at, e.g., 80% saturation. The resuspended and dialyzed precipitate is loaded onto a DEAE cellulose (DE 52) column and the bound proteins are eluted with a salt gradient (e.g., from 0.05-0.3 M NaCl) in 0.05 M Tris-HCl, pH 7.8. The “fold” purification of the protein can be calculated by measuring, for example, the GM-CSF activity of the heretofore unknown CSF.

The proteins isolated using DE 52 chromatography can then be further concentrated by dialyzing against 50% (w/v) polyethylene glycol in PBS and purified further by gel filtration. The final step in a purification scheme for isolating a CSF may involve chromatography on a reverse-phase HPLC (RP-HPLC) column. The majority of proteins do not bind to this column or elute at low 1-propanol concentrations (less than 20% 1-propanol). In an exemplary purification procedure, RP-HPLC can be performed with a Waters HPLC system (M 6,000 solvent delivery pumps, model 400 variable wavelength detector, data module and data processor, Waters, Associates, Milford, Mass.). The separation employs a uBondapak C18 column (Waters). Examples of buffers for the RP-HPLC purification are: Buffer A: 0.9 M acetic acid/0.2M pyridine, pH 4.0; buffer B: buffer A in 50% 1-propanol (Burdick, and Jackson Lab., Muskegon, Mich.). The CSF-containing pool obtained from gel filtration is acidified with acetic acid to pH 4.0 and injected onto the uBondapak C18 column without regard to sample volume. The column is washed with buffer A (10 minutes) and bound proteins are eluted using a steep gradient (0-40% buffer B for the first 20 minutes and a 40-100% gradient of buffer B for 120 minutes; at a flow rate of 1 ml/minute). Three ml fractions are collected for determination of CSF activity. From each fraction, an aliquot can be supplemented with 10% FCS, dialyzed against PBS, and tested for CSF activity.

The protein content of the HPLC fraction can be measured by comparing the density in silver-stained SDS-PAGE with protein standards of known concentrations. For example, the discontinuous Tris-glycine system of Laemmli (Laemmli, U.K., Nature, 227, 680-685 1970) can be used for 1.5 mm slab gels of 15% acrylamide. The samples (e.g., about 200 ng lyophilized protein eluted from HPLC) are treated with 1% SDS in 0.0625 M Tris-HCl, pH 6.8, at 37° C. for 1 hour under both reducing (5% 2-mercaptoethanol) and non-reducing conditions and then loaded onto a gel. After electrophoresis, gels are stained by the Biorad silver staining method (Biorad Laboratories, Rockville Centre, N.Y.). Apparent molecular weights are determined using protein standards.

After treatment of lyophilized CSF under non-reducing conditions and subsequent electrophoresis, parallel gels are sliced in 4 mm or 2 mm sections and proteins from each slice are eluted either into 0.5 ml RPMI 1640 containing 10% FCS or into phosphate-buffered saline (PBS; 20 mM phosphate, 0.15 M NaCl). After extensive dialysis, the eluted material is assayed for CSF activity and a specific activity of the isolated protein is obtained.

The final preparation obtained after HPLC can be subjected to isoelectric focusing to determine the isoelectric point of the protein. In order to do this, purified CSF from the HPLC fractionation is supplemented with 20% glycerol (v/v) and 2% ampholines (V/V), pH 3.5-10 (LKB Products, Inc.). A 5-60% glycerol density gradient containing 2% Ampholines, pH 3.5-10, is layered onto an isoelectrofocusing column (LKB 8100). The CSF sample is applied onto the isodense region of the gradient, followed by isoelectrofocusing (2,000 V, 24 hours). Five ml fractions are collected and the pH determined in each fraction. The fractions are dialyzed against PBS and subsequently tested for CSF activity.

The availability of purified human CSF derived from CD15⁺ hematopoietic progenitor cells has important and far reaching implications in the management of clinical diseases involving hematopoietic dysfunction or failure (i.e., hematopoietic abnormalities), either alone or in combination with other lymphokines or chemotherapy. Such abnormalities or disorders include leukemia and white cell disorders in general. Such factors will be useful in transplantation, whether allogeneic or autologous, to augment growth of bone marrow progenitor cells. These cells, in turn, can be used to treat induced forms of bone marrow aplasia or myelosuppression, in radiation therapy or chemotherapy-induced bone marrow depletion, wound healing, bum patients, and in bacterial inflammation, among other indications known in the art.

F. Polynucleotide Microarrays

DNA-based arrays provide a simple way to explore the expression of a single polymorphic gene or a large number of genes. In the invention, 5 to 15 or more nucleic acids having sequences selected from the sequences of SEQ ID NO: 1 through SEQ ID NO: 244 may be presented in a DNA microarray for the analysis and expression of these genes in various cell types. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These references are exemplary patents that disclose nucleic acid microarrays and those of skill in the art are aware of numerous other methods and compositions for producing microarrays.

The invention provides for a composition comprising a plurality of polynucleotide probes for use in detecting changes in expression of a large number of genes from CD15⁺ myeloid progenitor cells. As used herein, the term “polynucleotide probe” refers to a polynucleotide comprising any one of the nucleic acid sequences listed in SEQ ID NO: 1 through 244; or any fragment thereof. Preferably, the fragment is at least 5 nucleotides, preferably 9 nucleotides, and more preferably, at least 20 nucleotides. Such a composition can be employed for the diagnosis and/or treatment of any abnormality (i.e., condition, disorder or disease) in which the dysfunction or non-function of hematopoietic cells is implicated. Recognizing that hybridization results may vary depending upon the particular samples being assayed as well as expected variations in other variables known in the art, the methods of the invention are expected to provide useful diagnostic and/or therapeutic benefits with a rate of false results of at least 20%. That is, the methods are expected to be operable when up to 20% of the polynucleotide and/or oligonucleotide probes derived from SAGE tags either hybridize when they should not hybridize or fail to hybridize when they should hybridize. One of skill in the art, using the knowledge in the art and the disclosure herein, will be able to compensate for such failures by increasing the number of probes in the assay or by using controls to ensure usefully accurate results.

In one aspect, the invention provides a composition comprising a plurality of polynucleotide probes, wherein each of said polynucleotide probes comprises at least a portion of an expressed gene isolated from a population of CD15⁺ myeloid cells. More particularly, the plurality of polynucleotide probes each comprise at least a portion of one or more of the sequences (SEQ ID NOS: 1-244) presented in the Sequence Listing.

The composition is particularly useful as hybridizable array elements in a microarray for monitoring the expression of a plurality of target polynucleotides. The microarray comprises a substrate and the hybridizable array elements. The microarray is used, for example, in the diagnosis and/or treatment of a cancer, an immunopathology, a neuropathology, and the like.

Table 1 is a list of the known genes abnormally expressed in t(9;11) leukemic cells. Table 2 is a list of novel 3′ ESTS that were abnormally expressed in t(9;11) myeloid leukemia cells. Finally, Table 3 shows genes that were abnormally expressed in t(9;11) leukemic cells. For each of the SAGE tag sequences listed herein, it is understood that the sequence begins 5′-CATG, reflecting the NlaIII site added to each SAGE tag, as described above. One of skill will recognize that other sites may be used in place of, or in addition to, NlaIII.

The term “microarray” refers to an ordered arrangement of hybridizable array elements. The array elements are arranged so that there are preferably at least two or more different array elements, more preferably at least 100 array elements, and most preferably at least 1,000 array elements, on a 1 cm² substrate surface. The hybridization signal from each of the array elements is individually distinguishable. In a preferred embodiment, the array elements comprise polynucleotide probes.

A “polynucleotide” refers to a chain of nucleotides. Suitable lengths for polynucleotide probes according to the invention are polynucleotides of at least 8 nucleotides, and preferably at least 9, 10, 11, 12, 13, 14, 18, 20, or 25 nucleotides in length. Also preferred are chains having from about 75 to 10,000 nucleotides, more preferably from about 100 to 3,500 nucleotides. These preferred lengths can be conveniently generated from SAGE tags using GLGI, as described herein. The informational content of a polynucleotide probe is expected to be at least 6 nucleotides, and preferably at least 10 nucleotides or the physical length of the polynucleotide probe. An exemplary informational content of a polynucleotide probe is the 10 nucleotides of a typical SAGE tag, which has a typical physical length of 14 nucleotides, including 4 nucleotides derived from an anchor site. An “oligonucleotide” refers to a chain of nucleotides extending from 2-75 nucleotides, and preferably 9-79 nucleotides. The term “probe” refers to a polynucleotide sequence capable of hybridizing with a target sequence to form a polynucleotide probe/target complex. A “target polynucleotide” refers to a chain of nucteotides to which a polynucleotide probe can hybridize by base pairing. In some instances, the sequences will be complementary (no mismatches) when aligned. In other instances, there may be up to a 10% mismatch.

A “plurality” refers preferably to a group of at least 15 or more members, more preferably to a group of at least about 100, and even more preferably to a group of at least about 1,000, members. The maximum number of members is unlimited, but is at least about 100,000 members.

The term “gene” or “genes” refers to a polynucleotide sequence(s) of a gene which may be the partial or complete sequence and may comprise regulatory region(s), untranslated region(s), or coding regions. In one embodiment of the invention exemplified herein, the genes were initially identified from CD15⁺ myeloid progenitor cells.

The microarray can be used for large-scale genetic or gene expression analysis of a large number of target polynucleotides. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

When the composition of the invention is employed as hybridizable array elements in a microarray, the array elements are preferably organized in an ordered fashion so that each element is present at a distinguishable, and preferably specified, location on the substrate. In the preferred embodiments, because the array elements are at specified locations on the substrate, the hybridization patterns and intensities (which together create a unique expression profile) can be interpreted in terms of expression levels of particular genes and can be correlated with a particular disease or condition or treatment.

The composition comprising a plurality of polynucleotide probes can also be used to purify a subpopulation of mRNAs, cDNAs, genomic fragments and the like, in a sample. Typically, samples will include target polynucleotides of interest and other nucleic acids which may enhance the hybridization background; therefore, it may be advantageous to remove these nucleic acids from the sample. One method for removing the additional nucleic acids is by hybridizing the sample containing target polynucleotides with immobilized polynucleotide probes under hybridizing conditions. Those nucleic acids that do not hybridize to the polynucleotide probes are removed and may be subjected to analysis or discarded. At a later point, the immobilized target polynucleotide probes can be released in the form of purified target polynucleotides.

1. Microarray Production

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

In one embodiment, the probes are cDNAs. The size of the DNA sequence of interest may vary and is preferably from 100 to 10,000 nucleotides, more preferably from 150 to 3,500 nucleotides.

The probes can be prepared by a variety of synthetic or enzymatic schemes that are well known in the art. The probes can be synthesized, in whole or in part, using chemical methods well known in the art (Caruthers et al., Nucleic Acids Res., Symp. Ser., 7:215-233, 1980). Alternatively, the probes can be generated, in whole or in part, enzymatically.

A nucleotide analog can be incorporated into a probe by methods well known in the art. The only requirement is that the incorporated nucleotide analog must serve to base pair with a target polynucleotide sequence. For example, certain guanine nucleotides can be substituted with hypoxanthine, which base pairs with cytosine residues, even though these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups, although any known nucleotide modification is contemplated.

The polynucleotide probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillary tubes. The substrate can have a variety of surface forms, such as wells, trenches, pins, channels and pores, to which the polynucleotide probes are bound by any known form of attachment, but preferably by covalent bonding. Preferably, the substrates are optically transparent.

Polynucleotide probes, e.g., complementary DNA (cDNA), can be arranged and then immobilized on a substrate. The probes can be immobilized by covalent means such as by chemical bonding procedures or UV. In one such method, a cDNA is bound to a glass surface which has been modified to contain epoxide or aldehyde groups. In another case, a cDNA probe is placed on a polylysine-coated surface and then UV cross-linked (Shalon et al., PCT publication WO95/35505, herein incorporated by reference). In yet another method, a DNA is actively transported from a solution to a given position on a substrate by electrical means (Heller et al., U.S. Pat. No. 5,605,662). Alternatively, individual DNA clones can be gridded on a filter. Cells are lysed, proteins and cellular components degraded, and the DNA coupled to the filter by UV cross-linking.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

2. Sample Preparation for Microarray Analysis

In order to conduct sample analysis, a sample containing one or more target polynucleotides is provided. The samples can be any sample containing one or more target polynucleotides and obtained from any bodily fluid (blood, urine, saliva, phlegm, gastric juices, etc.), cultured cells, biopsies, or other tissue preparations.

DNA or RNA can be isolated from the sample according to any method known in the art. For example, methods of purification of nucleic acids are described in Tijssen, Laboratory Techniques in Biochemistry and Molecular Biology: Hybridization With Nucleic Acid Probes, Part I. Theory and Nucleic Acid Preparation, Elsevier, New York N.Y. 1993. In one case, total RNA is isolated using the TRIZOL reagent (Life Technologies, Gaithersburg Md.), and mRNA is isolated using oligo d(T) affinity purification using column chromatography or glass beads. Alternatively, when target polynucleotides are derived from an mRNA, the target polynucleotides can be a cDNA reverse-transcribed from an mRNA, an RNA transcribed from that cDNA, a DNA amplified from that cDNA, an RNA transcribed from the amplified DNA, and the like. When the target polynucleotide is derived from DNA, the target polynucleotide can be DNA amplified from DNA or RNA reverse transcribed from DNA. In yet another alternative, the targets are target polynucleotides prepared by more than one method.

When target polynucleotides are amplified, it is preferable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single-stranded DNA template. The second DNA strand is polymerized using a DNA polymerase and a RNase which assists in freeing the DNA from the DNA/RNA hybrid. After synthesis of the double-stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine, U.S. Pat. No. 5,514,545).

Quantitation controls may be included within the sample to assure that amplification and labeling procedures do not change the true distribution of target polynucleotides in a sample. For this purpose, a sample is spiked with a known amount of a control target polynucleotide and the composition of probes includes reference probes which specifically hybridize with the control target polynucleotides. After hybridization and processing, the hybridization signals obtained should accurately reflect the amounts of control target polynucleotide added to the sample.

Prior to hybridization, it may be desirable to fragment the nucleic acid target polynucleotides. Fragmentation improves hybridization by minimizing secondary structure and cross-hybridization to other nucleic acid target polynucleotides in the sample or to noncomplementary polynucleotide probes. Fragmentation can be performed by mechanical or chemical means.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³H, ¹⁴C, ³²P, ³³P or ³⁵S, chemiluminescent compounds. labeled binding proteins, heavy metal atoms, spectroscopic markers such as fluorescent markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes, and the like. Preferably, fluorescent markers absorb light above about 300 nn, preferably above 400 nm, and usually emit light at wavelengths at least greater than 10 nmn above or below the wavelength of the light absorbed. Preferred fluorescent markers include fluorescein, phycoerythrin, rhodamine, lissamine, and C3 and C5 available from Amersham Pharmacia Biotech (Piscataway N.J.).

Labeling can be carried out during an amplification reaction, such as polymerase chain reactions and in vitro transcription reactions, or by nick translation or 5′ or 3′-end-labeling reactions. When the label is incorporated after or without an amplification step, the label is typically incorporated by using terminal transferase or by phosphorylating the 5′ end of the target polynucleotide using, e.g., a kinase and then incubating overnight with a labeled oligonucleotide in the presence of T4 RNA ligase.

Alternatively, the labeling moiety can be incorporated after hybridization (i.e., once a probe/target complex has formed).

3. Use of Gene Sequences for Diagnostic Purposes

In certain embodiments, the polynucleotide sequences of any one of the polynucleotides comprising sequences set forth in any of SEQ ID NOs 1-244 may be used for the diagnosis of abnormalities (i.e., conditions or diseases) with which the abnormal expression of any one of those polynucleotides is associated. For example, a polynucleotide comprising any of the sequences set forth in SEQ ID NOs 1-244 may be used in hybridization or PCR assays of fluids or tissues from biopsies to detect abnormal gene expression. Such methods may be qualitative or quantitative in nature and may include Southern or Northern analyses, dot blot or other membrane-based technologies, PCR technologies, dip stick, pin, chip and ELISA technologies. All of these techniques are well known in the art and are the basis of many commercially available diagnostic kits.

In addition, such assays may be useful in evaluating the efficacy of a particular therapeutic treatment regimen in animal studies, in clinical trials, or in monitoring the treatment of an individual patient. In order to provide a basis for the diagnosis of disease, a normal or standard profile for the expression of a polynucleotide comprising any one of the sequences set forth in SEQ ID NOs 1-244 needs to be established. This generally involves a combination of body fluids or cell extracts taken from normal subjects, either animal or human, under conditions suitable for hybridization or amplification. Standard hybridization may be quantified by comparing the values obtained for normal subjects with a dilution series of a given leukemia-related polynucleotide comprising the sequence of any of SEQ ID NOs 1-244 run in the same experiment where a known amount of purified gene product is used. Standard values obtained from normal samples may be compared with values obtained from samples from cachectic subjects affected by abnormal gene expression in leukemic cells. Deviation between standard and subject values establishes the presence of disease.

Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

PCR, as described in U.S. Pat. Nos. 4,683,195 and 4,965,188, provides additional uses for oligonucleotides or polynucleotides comprising a sequence set forth as part of SEQ ID NOs 1-244. Such oligomers, or polymers, are generally chemically synthesized, but they may be generated enzymatically or produced from a recombinant source as described herein. Oligomers generally define two nucleotide sequences, one with sense orientation and one with antisense orientation, and a perfectly complementary pair of such oligomers may be employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Additionally, methods to quantitate the expression of a particular molecule include radiolabeling (Melby et al., J Immunol Methods 159: 235-44, 1993) or biotinylating (Duplaa et al., Anal Biochem 229-36, 1993) nucleotides, coamplification of a control nucleic acid, and standard curves providing a basis for interpolation, or extrapolation, of experimental results. Quantitation of multiple samples may be accelerated by running the assay in an ELISA format where the oligomer of interest is presented in various dilutions and a spectrophotometric or colorimetric response gives rapid quantitation. For example, the presence of abnormal levels of any one of the proteins encoded by the polynucleotides having a sequence set forth in any of SEQ ID NOs 1-244 in extracts of biopsied tissues may indicate the onset of a particular disease. A definitive diagnosis of this type may allow health professionals to begin aggressive treatment, thereby mitigating or ameliorating at least one symptom of the abnormality, e.g., condition. Similarly, further assays can be used to monitor the progress of a patient during treatment.

4. Hybridization and Detection in Microarrays

Hybridization causes a denatured probe and a denatured complementary target to form a stable nucleic acid duplex through base pairing. Hybridization methods are well known to those skilled in the art (See, e.g., Ausubel, Short Protocols in Molecular Biology, John Wiley & Sons, New York N.Y., units 2.8-2.11, 3.18-3.19 and 4-6-4.9, 1997). Conditions can be selected for hybridization where an exactly complementary target and probe can hybridize, i.e., each base pairs, or hydrogen bonds, with its complementary base. Alternatively, conditions can be selected where a target and a probe have mismatches but are still able to hybridize. Suitable conditions can be selected, for example, by varying the concentrations of salt in the prehybridization, hybridization and/or wash solutions, by varying the hybridization and wash temperatures, or by varying the polarity of the prehybridization, hybridization and/or wash solutions.

Hybridization can be performed at low stringency with buffers, such as 6×SSPE with 0.005% Triton X-100 at 37° C., which permits hybridization between target and probes that contain some mismatches to form target polynucleotide/probe complexes. Subsequent washes are performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC, Background signals can be reduced by the use of detergent, such as sodium dodecyl sulfate, Sarcosyl or Triton X-100, or a blocking agent, such as salmon sperm DNA.

After hybridization, the microarray is washed to remove nonhybridized nucleic acids, and complex formation between the hybridizable-array elements and the target polynucleotides is detected. Methods for detecting complex formation are known in the art. In a preferred embodiment, the target polynucleotides are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensity. The scanned image is examined to determine the abundance/expression level of each hybridized target polynucleotide.

Typically, microarray fluorescence intensities can be normalized to take into account variations in hybridization intensities when more than one microarray is used under similar test conditions. In a preferred embodiment, individual probe/target hybridization intensities are normalized using the intensities derived from internal normalization controls contained on each microarray.

5. Microarray Expression Profiles

This section describes an expression profile using the composition of the invention. The expression profile can be used to detect changes in the expression of genes implicated in abnormalities such as disease.

The expression profile includes a plurality of detectable complexes. Each complex is formed by hybridization of one or more nucleic acids of the invention to one or more complementary target polynucleotides. At least one of the nucleic acids of the invention, and preferably a plurality thereof, is hybridized to a complementary target polynucleotide forming at least one, and preferably a plurality, of complexes. A complex is detected by incorporating at least one labeling moiety in the complex as described above. The expression profiles provide “snapshots” that can show unique expression patterns that are characteristic of the presence or absence of a disease or condition.

After performing hybridization experiments and interpreting detected signals from a microarray, particular probes can be identified and selected based on their expression patterns. Such probe can be used to clone a full-length gene or to produce a polypeptide.

The composition comprising a plurality of probes can be used as hybridizable elements in a microarray. Such a microarray can be employed in several applications including diagnostics, prognostics and treatment regimens, drug discovery and development, toxicological and carcinogenicity studies, forensics, pharmacogenomics, and the like.

In one situation, the microarray is used to monitor the progression of disease. Researchers can assess and catalog the differences in gene expression between healthy and diseased tissues or cells. By analyzing changes in patterns of gene expression, disease can be diagnosed at earlier stages, before the patient is symptomatic. The invention can also be used to monitor the efficacy of treatment. For some treatments with known side effects, the microarray is employed to “fine tune” the treatment regimen. A dosage is established that causes a change in genetic expression patterns indicative of successful treatment. Expression patterns associated with undesirable side effects are avoided. This approach may be more sensitive and rapid than waiting for the patient to show inadequate improvement, or to manifest side effects, before altering the course of treatment.

Alternatively, animal models which mimic a disease, rather than patients, can be used to characterize expression profiles associated with a particular abnormality, such as a disease or condition. This gene expression data is useful in diagnosing and monitoring, e.g., the course of disease in a patient, in determining gene targets for intervention, and in testing treatment regimens.

Also, the microarray is useful in rapidly screening large numbers of candidate drug molecules, looking for ones that produce an expression profile similar to those of known therapeutic drugs, with the expectation that molecules with the same expression profile will have similar therapeutic effects. Thus, the invention provides the means to determine the molecular mode of action of a drug.

G. Therapeutic and Pharmaceutical Aspects of the Invention

The invention has been disclosed as providing, in part, various genes identified from CD15⁺ myeloid progenitor cells and assays for determining the nature and level of activity of the encoded gene products, such as CSF. It is contemplated that CSF factors of the invention will be used in the intervention of various disease states such as, for example, clinical diseases involving hematopoietic abnormalities (i.e., dysfunction or failure), either alone or in combination with other lymphokines or chemotherapy (e.g., leukemia and white cell disorders). When used in such therapeutic applications, it is preferable to prepare the compositions of the invention in pharmaceutically acceptable formats (i.e., as pharmaceutical compositions), for example by combining the therapeutic composition with a pharmaceutically acceptable adjuvant, carrier or diluent.

A particular use for the polynucleotide (e.g., genes) of the invention is in autologous bone marrow transplants for individuals suffering from bone marrow aplasia or myelosuppression such as that seen in response to radiation therapy or chemotherapy.

Autologous bone marrow transplant (ABMT) is an example of ex vivo gene therapy. In ex vivo therapy, cells from the patient are removed and maintained outside the body for a period of time. During this period, a gene is introduced into the cells, after which the cells are reintroduced into the patient. In ABMT, the patient will serve as his/her own bone marrow donor. Thus, a normally lethal dose of irradiation or chemotherapy may be delivered to the patient to kill tumor cells, and the bone marrow repopulated with the patients own cells that have been maintained (and perhaps expanded) ex vivo.

H. Method of Treating Leukemia in Humans with the Therapeutic Compositions of the Invention.

The described therapy methods may be used for the processing and purging of bone marrow samples of individuals having myeloid leukemia. In one embodiment, the tissue is treated with a therapy employing a composition comprising any sequence set forth in SEQ ID NOs 1-244; the treated tissue may be reintroduced into the leukemic animal as an autologous transplant. As such, a therapeutic tool to treat a patient with leukemia is provided.

As part of a total clinical treatment protocol for a patient, the method preferably should provide at least a two-log (100-fold) reduction in the ratio of leukemic cells to normal cells, in addition to the 10,000/1 to 1/1 reduction (from chemotherapy) and the 2-log reduction (from fractionation of the marrow cells subsequent thereto) of leukemia cells to normal cells which may be achieved with conventional treatment regimens with non-pre-treated bone marrow tissue transplants. The proposed regimen thereby effectively reduces the number of leukemia cells in the patient to levels which enhance the therapeutic index of the bone-marrow transplant treatment. In some cases, up to a 3-log (1000-fold) increase in the number of normal, non-leukemic cells in a patient's bone marrow cell population is achievable upon the reintroduction of a pre-treated bone marrow sample.

The reintroduction of a patient's pre-treated autologous bone marrow sample also offers a method for treating CML disease and for preventing the transition of leukemia from its chronic phase to the more serious forms of acute leukemia.

A processed autologous bone marrow sample prepared as described herein is used as part of a total leukemia treatment regimen. Once a prepared bone marrow sample is processed according to the methods disclosed herein, standard protocols employed for the general technique of performing a bone marrow transplant in CML may be used to obtain an initial bone marrow sample and to reintroduce the processed bone marrow to the patient. Such general clinical techniques are described by Canaani et al., Lancet 1(8377):593-595 (11984), which reference is specifically incorporated herein by reference for this purpose. A volume of about 50-100 cc of purified marrow (containing about 2.5×10⁷ cells) is the volume of processed bone marrow tissue which will be given to the patient to effect the claimed treatment.

A proposed method for treating leukemia in a patient according to the invention comprises. identifying a patient having leukemia; administering to the identified patient a chemotherapeutic regimen sufficient to generate cytogenetic remission in the patient in the ratio of leukemia cells to normal cells; obtaining a bone marrow sample from the identified patient in cytogenetic remission; exposing the bone marrow sample from the patient in cytogenetic remission to a therapeutic composition derived from the invention to provide an essentially leukemia-free bone marrow sample; and reintroducing the essentially leukemia-free bone marrow sample into the identified patient, wherein the reintroduction of the sample provides replacement of leukemic marrow cells with normal hematopoietic progenitor cells in a method for treating leukemia.

Also, it should be understood that it may be that purified CSFs may be routinely prepared into pharmaceutically acceptable forms of the proteins once they are isolated from the media and/or cellular compositions described above. Generally, this will entail preparing compositions that are essentially free of pyrogens, as well as other impurities that could be harmful to humans or animals.

One will generally desire to employ appropriate salts and buffers to render the compositions stable and allow for uptake by target cells. Buffers also will be employed when recombinant cells are introduced into a patient. The phrase “pharmaceutically or pharmacologically acceptable” refers to molecular entities and compositions that do not produce unacceptably adverse, allergic, or other untoward reactions when administered to an animal or a human. As used herein, “pharmaceutically acceptable carrier” may include any and all solvents, dispersion media, coatings, antibacterial and antifungal agents, isotonic and absorption delaying agents, excipients, adjuvants, carriers, and the like. The use of such media and agents for pharmaceutically active substances is well known in the art. Except insofar as any conventional media or agent is incompatible with the gene or CSF compositions produced by the invention, its use in therapeutic compositions is contemplated. Supplementary active ingredients also can be incorporated into the compositions.

The compositions of the invention include classic pharmaceutical preparations. Administration of these compositions according to the invention will be via any common route so long as the target tissue is available via that route. The pharmaceutical compositions may be introduced into the subject by any conventional method, e.g., by intravenous, intradermal, intramuscular, intramammary. intraperitoneal, intrathecal, intraocular, retrobulbar, intrapulmonary (e.g., term release), oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The compositions produced using the invention may be prepared for administration as solutions of the free compound (e.g., a free base) or pharmacologically acceptable salts in water suitably mixed with a surfactant, such as hydroxypropylcellulose. Dispersions also can be prepared in glycerol, liquid polyethylene glycols and mixtures thereof, and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that functional syringability is retained. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum mono,stearate and gelatin.

Sterile injectable solutions are prepared by incorporating the active compounds in an effective amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients selected from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents, excipients, adjuvants, carriers, and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the compositions.

For oral administration, the compositions produced by the invention may be combined with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including gels, pastes, powders, and slurries. The active ingredient may be added in a therapeutically effective amount to a paste dentifrice that may include water, binders, abrasives, flavoring agents, foaming agents, and humectants.

The compositions of the invention may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine, and the like.

Upon formulation, solutions will be administered in a manner compatible with the dosage formulation and in such amount as is therapeutically effective. The formulations are easily administered in a variety of dosage forms such as injectable solutions, drug release capsules, and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990, Mack Publ. Co, Easton Pa. 18042) pp. 1435-1712, incorporated herein by reference. Such formulations may influence the physical state, stability, rate of in vivo release and rate of in vivo clearance of the administered agents. Depending on the route of administration, a suitable dose may be calculated according to body weight, body surface area(s), or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose-response data. The final dosage regimen will be determined by the attending physician, considering factors which modify the action of drugs, e.g., the drug's specific activity, severity of the damage and the responsiveness of the patient, the age, condition, body weight, sex, and diet of the patient, the severity of any infection, time of administration, and other clinical factors.

It will be appreciated that the pharmaceutical compositions and treatment methods,employing such compositions may be useful in fields of human medicine and veterinary medicine. Thus, the subject to be treated may be a mammal, preferably a human or other animal. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs, and hamsters; and poultry such as chickens, turkey, ducks and geese.

### I. EXAMPLES

The following examples present preferred embodiments and techniques, but are not intended to be limiting. Those of skill in the art will, in light of the present disclosure, appreciate that many changes can be made in the specific materials and methods which are disclosed and still obtain a like or similar result without departing from the spirit and scope of the invention. In the following examples, Example 1 provides the materials and methods used in developing and refining the invention; Example 2 describes the distribution of SAGE tags in CD15⁺ cells; and Example 3 discloses functional analyses of the polynucleotides having sequences set forth in SEQ ID NOS: 1-244.

### Example 1

Materials and Methods

Cell purification. Human bone-marrow mononuclear cells were obtained from the Poietics Company (Gaithersburg, Md.). These cells were isolated from bone marrow with Ficoll/Paque solution and stored in liquid nitrogen. Cells from three donors were thawed at 37° C., pooled, and used immediately for the isolation. of myeloid progenitor cells with CD15⁺ magnetic beads (Dynal., Oslo, Norway) according to the manufacturer's protocol. The purity of isolated cells was greater than 95%, as confirmed by fluorescence-activated cell sorter analysis.

In order to detect the expression of MLL-AF9 fusion transcripts, RT-PCR reactions with primers hybridizing to the 5′ end of the MLL gene and the 3′ end of the AF9 gene were performed. The amplified products were cloned-and sequenced. Anchored oligo dT primers, 5′ biotin-ATCTAGAGCGGCCGC-T16-A/G (SEQ ID NO: 313), 5′ biotin-ATCTAGAGCGGCCGC-T16-C-A/G/C (SEQ ID NO: 314), were used for generation of cDNA and the 3′ part of the anchored primers 5′-ACTATCTAGAGCGGCCGCTT-3′ (SEQ ID NO: 315) served as a universal antisense primer for GLGI.

SAGE. Serial Analysis of Gene Expression (SAGE) is a powerful method for obtaining comprehensive and quantitative gene expression profiles from cell populations under selected morphological and physiological conditions (Velculescu, et al., Science, 270, 484-487, 1995; Velculescu, et al., Cell, 88, 243-251, 1997; Porter, et al., Cancer Res., 61, 5697-5702, 2001; Gunnerson, et al., Glia, 32, 146-154, 2000; Jones, et al., Genome Res., 11, 1345-1352, 2001; Matsumura, et al., Plant J., 20, 719-726, 1999). In the SAGE technique, short sequence tags of about 10 nucleotides, each representing an expressed sequence, are excised and the tag from different expressed sequences are ligated for sequencing analysis. Essentially, the SAGE technique quantifies a sequence “tag” that represents the transcription product of a gene. The SAGE technique provides maximal coverage of the expressed genes for gene identification at the whole genome level.

SAGE was performed with a modified SAGE protocol (Lee et al., BioTechniques, 31, 348-354, 2001, incorporated herein by reference), and the data were processed by use of the procedure described by Lee et al., Proc. Natl. Acad. Sci. (USA), 98, 3340-3345, 2001), incorporated herein by reference.

GLGI. The Generation of long cDNA from SAGE Tags for Gene Identification (GLGI) can convert the SAGE tags of 10 bases into their corresponding 3′ cDNA fragments covering several hundred bases (Chen, et al., Proc. Natl. Acad. Sci., 97, 349-353, 2000). With more sequence information, the genes can be correctly identified and characterized.

The GLGI method provides two advantages when conducting a broad-based analysis of cellular gene expression. First, one can identify the correct sequence from multiple sequences matched by a single SAGE tag. Second, one can generate a longer 3′ EST from a SAGE tag that does not match any known sequence, thereby furthering the analysis of the entire unknown expression unit (Chen et al., Proc. Natl. Acad. Sci. (USA), 97, 349-353, 2000). As described above, the GLGI process uses a SAGE tag sequence as the sense primer and an anchored oligo dT primer as the antisense primer to amplify the original cDNA template from which the SAGE tag was derived. GLGI has been developed into a high-throughput procedure for the large-scale conversion of SAGE tags into 3′ ESTs (Chen et al., Genes, Chromosomes & Cancer, 2001 (in press)). In the Examples described in the present application, the high-throughput GLGI procedure was used to identify precisely the expressed sequences corresponding to multi-matched SAGE tags found in more than 9 copies, and to convert novel SAGE tag sequences into 3′ ESTs for SAGE tags found in more than 4 copies.

### Example 2

Distribution of SAGE Tags in CD15⁺ Cells

SAGE libraries. Separate SAGE libraries were generated from one normal and three t(9;11) patient samples. For the generation of SAGE libraries, the procedure described above and in Lee et al., Proc. Natl. Acad. Sci. (USA), 98, 3340-3345, 2001, incorporated herein by reference, was applied. With the exception of the normal myeloid progenitor cell library, 3,000 clones were sequenced from each library. The extraction of SAGE tags from the collected sequences was performed with SAGE 300 software. A total of 37,519 unique SAGE tags were identified from 100,946 individual SAGE tags from the normal myeloid progenitor cells. A total of 29,852; 18,487; and 19,582 unique SAGE tags were identified from 67,828 SAGE tags of one AML M5, 55,219 SAGE tags of another AML M5, and 54,692 SAGE tags of one M1 sample, respectively. A total of 77,188 unique tags were identified from 278,685 individual tags that were extracted from one normal and three leukemic samples. Table 5 summarizes these findings.

TABLE 5

| | NORMAL | M5A | M5B | M1 |
|---|---|---|---|---|
| Total Tags | 100,946 | 67,828 | 55,219 | 54,692 |
| Unique Tags | 37,519 | 29,864 | 18,496 | 19,581 |

Candidate tags that are differentially expressed in leukemic compared to normal cells. The SAGE tag data from each of the t(9;11) samples were compared, and a set of consensus SAGE tags was developed. This consensus set was compared to data from normal CD15⁺ cells. A total of 244 tags showed differences in gene expression over five fold, which provided a sufficient number of tags to distinguish leukemic from normal cells. Accordingly, we used a threshold level of a five-fold change in expression, although it is expected that any detectable change in expression, such as a two-fold change, would suffice. Of these 223 tags, 68 tags were novel and 155 tags were known genes or ESTs. The novel tags were converted to 3′ ESTs by using GLGI. The amplified products were cloned and sequenced. The sequences were compared to known sequences available in public databases, such, as the NCBI database. The known gene lists and 3′ ESTs converted from novel tags are shown in Tables 1 and 2, respectively. Interestingly, more genes were down-regulated or turned off in the t(9;11) leukemic samples compared with up-regulated or turned-on genes in those samples. Table 6 summarizes these findings.

TABLE 6

| | Known | Novel | Unconfirmed | Total |
|---|---|---|---|---|
| Up-regulated | 24 | 5 | 4 | 33 |
| Down-regulated | 82 | 36 | 8 | 126 |
| Turned-on | 10 | 2 | 2 | 14 |
| Turned-off | 39 | 25 | 7 | 71 |
| Total | 155 | 68 | 21 | 244 |

### Example 3

Functional Analysis of the Genes Identified in Human Hematopoietic CD15⁺ Stem/Progenitor Cells

Hematopoietic differentiation is tightly controlled by different genes expressed in a temporal program. Abnormal differentiation can lead to the development of various diseases such as leukemia. As described above, 244 genes were found to be abnormally expressed in human leukemic cells.

Human embryonic stem (ES) cell lines have been established by several laboratories (e.g., Thomson et al., Trends Biotechnol. 18(2):53-57, 2000; Reubinoff et al., Nat. Biotech., 18, 399-404, 2000). These ES cells were derived from human blastocysts. Human ES cells are able to differentiate into embryoid bodies comprising the three embryonic germ layers (Itskovitz-Eldor et al., Mol. Med. 6, 88-95, 2000). Exploitation of ES cell biology potentially provides a solution to the major obstacles for generic gene therapy, given the demonstrated ability of such cells to differentiate into adult precursor and differentiated cell type in vitro, and the cell's suitability for precise genomic modification (Rathjen et al., Reprod. Fertil. Dev., 10, 31-47, 1998).

It has been reported that human ES cells have the potential to differentiate into hematopoietic cells (Odorico et al., Stem Cells, 19(3):193-204, 2001). Cells differentiating under specific and known culture conditions have typical hematopoietic features, such as the CD15⁺ marker. These cells can differentiate further into a variety of hematopoietic cells, including erythroid, macrophage, granulocyte, and megakaryocyte cells. Schuldiner et al., Proc. Natl. Acad. Sci. USA, 97(21):11307-12, 2000, incorporated herein by reference, established conditions for use with different growth factors to differentiate human ES cells, including basic fibroblast growth factor (bFGF), transforming growth factor-beta (TGF-beta), activin-A, bone morphogenic protein 4 (BMP-4), hepatocyte growth factor (HGF), epidermal growth factor (EGF), beta nerve growth factor (betaNGF), and retinoic acid.

RNAi is a powerful technique recently developed for gene “knock-down.” In this method, a synthesized 21-base double-stranded RNA is transfected into a mammalian cell. This double-stranded RNA will bind to a target endogenous expressed transcript, which will then be degraded by RNases. The advantage of this technique is that one needs only to have information of 21 bases of the targeted gene. There is no need to have other information for this targeted gene, e.g., genomic location, full-length sequences, and the like.

Human and mouse ES cell systems and the RNAi technique can be used to investigate the hematopoietic functions of the genes identified above. In an exemplary technique for identifying the function of any of the genes, one performs the following analysis: 1) isolate the hematopoietic CD15⁺ stem cells from a mouse ES cell line; 2) introduce the genes into the cells using the RNAi technique (the 21-base double-strand RNA can be designed for each of the novel ESTs to knock down the targeted genes expressed in the ES cells); and 3) monitor the phenotypic changes of the transfected cells using FACS analysis with, e.g., specific markers for T cells, B cells, myeloid cells, monocytes, dendrite cells, and macrophages, among other cell types.

Comparative analyses using control untransfected cells would identify the changes of these markers in the transfected cells. Any difference in the phenotypes in the transfected cells compared to the controls indicates that the targeted genes play a role in hematopoietic differentiation. By monitoring the distribution of the specific markers representing a specific lineage, the function of the targeted genes is identified and a determination of the lineages involving these genes is made.

The above assays can be conducted in the mouse D3 ES cell system. In addition, similar analyses may be performed in human ES cells. Human ES cells may be obtained from commercial suppliers, known to those of skill in the art, such as the Monash Institute of Reproduction & Development, Clayton, Australia, and the University of Wisconsin.

While the invention has been described in terms of specific embodiments, it is understood that variations and modifications will occur to those skilled in the art. Accordingly, only those limitations appearing in the appended claims should be placed upon the invention.

TABLE 1

| Known genes abnormally expressed in t(9;11) leukemic cells | | | | | | | | | |
|---|---|---|---|---|---|---|---|---|---|
| Normal | M1 | M5 | M5 | Expression | SEQ ID NO. | SAGE Tag | Gene | UniGene ID | Genbank ID |
| 26 | 0 | 2 | 2 | DN | 1 | CTGACTTGTG | major histocompatibility complex, class I, B | Hs.77961 | M12678 |
| 25 | 0 | 5 | 0 | DN | 2 | ACTTTAATGA | complement component 5 receptor 1 (C5a ligand) | Hs.2161 | NM_001736 |
| 21 | 0 | 1 | 1 | DN | 3 | GAGTAAAAAA | phosphatidylserine-specific phospholipase A1 alpha | Hs.17752 | AF035268 |
| 21 | 0 | 0 | 3 | DN | 4 | CTTTTTTCCC | CD48 antigen (B-cell membrane protein) | Hs.901 | M59904 |
| 20 | 1 | 4 | 1 | DN | 5 | GCTCGCCTTT | mycloperoxidase | Hs.1817 | M19507 |
| 18 | 3 | 2 | 1 | DN | 6 | GGTACCCATT | dual specificity phosphatase 6 | Hs.180383 | BC003143 |
| 18 | 0 | 0 | 1 | DN | 7 | CTGAGAAACT | ferritin, heavy polypeptide 1 | Hs.62954 | AA708260 |
| 15 | 0 | 0 | 1 | DN | 8 | GTGTGTTTGT | transforming growth factor, beta-induced, 68 kD | Hs.118787 | M77349 |
| 14 | 0 | 2 | 0 | DN | 9 | GTCGTAGCAC | peptidyl arginine deiminase, type V | Hs.117232 | NM_012387 |
| 13 | 2 | 0 | 0 | DN | 10 | CTGTACATAC | NS1-binding protein | Hs.197298 | NM_016389 |
| 13 | 1 | 1 | 1 | DN | 11 | TCACGTTAGG | integral membrane protein 2B | Hs.239625 | AF246221 |
| 13 | 0 | 2 | 2 | DN | 12 | GGCCAGGACT | formyl peptide receptor 1 | Hs.753 | L10820 |
| 11 | 0 | 0 | 1 | DN | 13 | AGGTCTTCAA | Thrombospondin 1 | Hs.87409 | X14787 |
| 10 | 2 | 2 | 0 | DN | 14 | AGCCTCGGCC | Rho guanine nucleotide exchange factor (GEF) 1 | Hs.252280 | NM_004706 |
| 10 | 1 | 0 | 2 | DN | 15 | CAATTTGTGT | interleukin 1,beta | Hs.126256 | M15330 |
| 9 | 1 | 0 | 2 | DN | 16 | AGTTATTTCT | ARP2 (actin-related protein 2, yeast) homolog | Hs.42915 | NM_005722 |
| 9 | 0 | 2 | 0 | DN | 17 | CTAATATTGT | RGC32 protein | Hs.76640 | AF036549 |
| 8 | 1 | 1 | 1 | DN | 18 | GAGGGGATGT | RAB31, member RAS oncogene family | Hs.223025 | NM_006868 |
| 8 | 1 | 0 | 1 | DN | 19 | TTATTGGGTA | FYN-binding protein (FYB-120/130) | Hs.58435 | AF001862 |
| 8 | 1 | 0 | 0 | DN | 20 | GGCTTTGGGG | CGI-10 protein | Hs.12239 | AF132944 |
| 7 | 1 | 1 | 1 | DN | 21 | CTTTTCAAGA | membrane cofactor protein (CD46) | Hs.83532 | X59408 |
| 7 | 1 | 1 | 1 | DN | 22 | TGTCCACACA | Homo sapiens mRNA; cDNA DKFZp586P1622 | Hs.5897 | AL110207 |
| 7 | 1 | 1 | 0 | DN | 23 | ACTTCACAAA | hypothetical protein FLJ22405 | Hs.27556 | NM_022485 |
| 6 | 1 | 0 | 0 | DN | 24 | GAACGTCTTA | SON DNA binding protein | Hs.92909 | X63751 |
| 6 | 0 | 0 | 1 | DN | 25 | CAAGCAAAAT | nitrogen fixation cluster-like | Hs.9908 | U47101 |
| 6 | 1 | 1 | 1 | DN | 26 | AAGATGCACA | autotaxin | Hs.174185 | L35594 |
| 6 | 0 | 1 | 1 | DN | 27 | TCAGATGGCG | tumor protein D52-like 2 | Hs.154718 | NM_003288 |
| 6 | 0 | 1 | 0 | DN | 28 | CTCAACCCCC | low density lipoprotein-related protein 1 | Hs.89137 | NM_002332 |
| 6 | 0 | 1 | 0 | DN | 29 | GACTGGACCG | found in inflammatory zone 3 | Hs.283091 | NM_020415 |
| 6 | 0 | 1 | 0 | DN | 30 | TGGTCATACT | Fc fragment of IgG, low affinity IIa, receptor for (CD32) | Hs.788641 | Y00644 |
| 5 | 0 | 0 | 1 | DN | 31 | AGGACAGAAG | matrix metalloproteinase 25 | Hs.198265 | AJ239053 |
| 5 | 1 | 0 | 1 | DN | 32 | AGAAATAAAG | adenosine monophosphate deaminase 2 (isoform L) | Hs.82927 | M91029 |
| 5 | 1 | 0 | 0 | DN | 33 | CACCACGGTG | RNB6 | Hs.241471 | AF052504 |
| 5 | 1 | 1 | 1 | DN | 34 | CGTGGAGCAA | HSPC171 protein | Hs.279593 | AF161519 |
| 5 | 0 | 1 | 1 | DN | 35 | GGAAGGCAGT | hypothetical protein FLJ11457 | Hs.126707 | NM_024753 |
| 5 | 1 | 0 | 0 | DN | 36 | GGGCACAATG | glycoprotein 1b (platelet), beta polypeptide | Hs.283743 | NM_000407 |
| 5 | 0 | 1 | 0 | DN | 37 | TATATATACA | hypothetical protein MGC16823 | Hs.238730 | BC007203 |
| 5 | 1 | 1 | 0 | DN | 38 | AGATAATCAG | full length insert cDNA clone EUROIMAGE 254679 | Hs.284310 | AL109708 |
| 5 | 1 | 1 | 0 | DN | 39 | CATTAAAGTA | Homo sapiens cDNA FLJ14296 fis, clone PLACE1008455 | Hs.288581 | AK024358 |
| 5 | 1 | 0 | 1 | DN | 40 | CCCACACTAC | guanine nucleotide binding protein, beta polypeptide 2 | Hs.91299 | M16538 |
| 5 | 1 | 0 | 1 | DN | 41 | GATGTTATTG | gastric inhibitory polypeptide receptor | Hs.251412 | S79852 |
| 5 | 1 | 0 | 1 | DN | 42 | TACACACGGA | Similar to putative nuclear protein | Hs.40342 | BC003618 |
| 5 | 0 | 1 | 0 | DN | 43 | AATCAATGTC | plasminogen activator, urokinase receptor | Hs.179657 | AC006953 |
| 5 | 0 | 1 | 0 | DN | 44 | ACGGTCCAGG | cytidine deaminase | Hs.72924 | L27943 |
| 5 | 1 | 0 | 0 | DN | 45 | GGCTCATCTT | myeloid cell leukemia sequence 1 (MCL1) | Hs.86386 | AF118124 |
| 5 | 1 | 0 | 0 | DN | 46 | TCCTTAGGCT | erythroid differentiation-related factor | Hs.274309 | AF364517 |
| 28 | 0 | 1 | 0 | OFF | 47 | GGAAAAGTGG | Serine (or cysteine) proteinase inhibitor, clade A | Hs.297681 | NM_000295 |
| 14 | 0 | 0 | 0 | OFF | 48 | GCCAAAAACC | peptidoglycan recognition protein | Hs.137583 | AF076483 |
| 12 | 0 | 0 | 0 | OFF | 49 | GTTATAATAC | serum/glucocorticoid regulated kinase | Hs.296323 | AF153609 |
| 11 | 0 | 0 | 0 | OFF | 50 | ACATTTCCAA | Putative lymphocyte G0/G1 switch gene | Hs.95910 | M69199 |
| 9 | 0 | 0 | 0 | OFF | 51 | ATCTTGAAAG | nucleosome assembly protein 1-like 1 | Hs.179662 | NM_004537 |
| 7 | 0 | 0 | 0 | OFF | 52 | TGAGCCAAAT | granule protein (28 kDa); cysteine-rich secretory protein-3 | Hs.54431 | X95240 |
| 7 | 0 | 0 | 0 | OFF | 53 | CTGAAGCTAA | DKFZP564A032 protein | Hs.23889 | NM_015474 |
| 7 | 0 | 0 | 0 | OFF | 54 | GCAAAACAAC | lactotransferrin | Hs.105938 | M93150 |
| 6 | 0 | 0 | 0 | OFF | 55 | GTGACCACAG | Rhesus blood group-associated glycoprotein | Hs.169536 | AF031549 |
| 6 | 0 | 0 | 0 | OFF | 56 | GAACTGGAGA | SH3-domain binding protein S (BTK-associated) | Hs.109150 | BC010123 |
| 6 | 0 | 0 | 0 | OFF | 57 | GCCTGCTATT | defensin, alpha 1, myeloid-relatcd sequence | Hs.274463 | NM_004084 |
| 5 | 0 | 0 | 0 | OFF | 58 | CCAGCGGCTG | MIL1 protein isoform 1 | Hs.10267 | AF246665 |
| 5 | 0 | 0 | 0 | OFF | 59 | TATCTGTATA | hypothetical protein | Hs.91165 | NM_019018 |
| 5 | 0 | 0 | 0 | OFF | 60 | AAACGCTACT | granzyine B | Hs.1051 | M28879 |
| 5 | 0 | 0 | 0 | OFF | 61 | ATGAGACCCT | Homo sapiens clone 23676 mRNA sequence | Hs.100841 | AF035278 |
| 5 | 0 | 0 | 0 | OFF | 62 | GGAAGAGTGC | myeloid cell differentiation protein (MCL1) gene | Hs.86386 | AF147742 |
| 5 | 0 | 0 | 0 | OFF | 63 | TAGTTTGAAG | glutaminyl-peptide cyclotransferase (glutaminyl cyclase) | Hs.79033 X71125 | |
| 5 | 0 | 0 | 0 | OFF | 64 | TATGAGGAGG | matrix metalloproteinase 25 | Hs.198265 | NM_022468 |
| 0 | 34 | 11 | 52 | ON | 65 | GAAGGTGATC | XAGE-1 protein | Hs.112208 | AF251237 |
| 0 | 12 | 5 | 13 | ON | 66 | GGCAGAGGAC | non-metastatic cells 1, protein (NM23A) expressed in | Hs.118638 | BC000293 |
| 0 | 9 | 13 | 5 | ON | 67 | CAGTCTTTTG | Home sapiens mRNA; cDNA DKFZp434K052 | Hs.169639 | AL137398 |
| 0 | 13 | 9 | 6 | ON | 68 | TGAAAGTGTG | KIAA0201 gene | Hs.36927 | D86956 |
| 0 | 8 | 5 | 11 | ON | 69 | GAAATCCGCA | mannosidase, alpha, class 2B, member 1 | Hs.279854 | BC000736 |
| 0 | 5 | 7 | 8 | ON | 70 | CCACCCCGAA | testis enhanced gene transcript (BAX inhibitor 1) | Hs.74637 | BC000916 |
| 0 | 8 | 5 | 5 | ON | 71 | TTTAAAACTT | EXP35 mRNA | Hs.28578 | AF255334 |
| 0 | 5 | 5 | 5 | ON | 72 | CGACTCTGGC | glucosidase I | Hs.83919 | NM_006302 |
| 9 | 55 | 52 | 40 | UP | 73 | AGGTCCTAGC | glutathione S-transferase pi | Hs.226795 | U30897 |
| 5 | 52 | 27 | 33 | UP | 74 | AACCTGGGAG | KIAA0563 gene | Hs.200597 | NM_014834 |
| 5 | 27 | 30 | 29 | UP | 75 | GGAAGAGCTG | Williams-Beuren syndrome chromosome region 5 | Hs.56607 | AF252611 |
| 2 | 20 | 14 | 18 | UP | 76 | AGAATTGCTT | nephrin (NPHS1) | Hs.190311 | AF035835 |
| 2 | 24 | 10 | 14 | UP | 77 | CATATCATTA | insulin-like growth factor binding protein 7 | Hs.119206 | NM_001553 |
| 2 | 9 | 9 | 22 | UP | 78 | TAATTCTTCT | chaperonin containing TCP1, subunit 3 (gamma) | Hs.1708 | BC006501 |
| 2 | 10 | 11 | 12 | UP | 79 | AAGATCCCCG | divalent cation tolerant protein CUTA | Hs.107187 | AF106943 |
| 1 | 25 | 10 | 32 | UP | 80 | AATTTTATTT | poly(rC)-binding protein 1 | Hs.2853 | NM_006196 |
| 1 | 39 | 7 | 26 | UP | 81 | GACAGAGTCC | LDL-related protein-associated protein 1 | Hs.75140 | NM_002337 |
| 1 | 5 | 5 | 26 | UP | 82 | TGGTTGATTT | KIAA0906 protein | Hs.56966 | AB020713 |
| 1 | 5 | 8 | 16 | UP | 83 | ACCCTTCCCT | odorant-binding protein 2B (OBP2B) | Hs.99526 | NM_014581 |
| 1 | 8 | 7 | 7 | UP | 84 | GTTACAAACT | topoisomerase (DNA) II beta (180 kD) | Hs.75248 | U54831 |
| 1 | 12 | 12 | 11 | UP | 85 | CTTTGATGTT | WD repeat domain 1 | Hs.85100 | AF020260 |
| 1 | 9 | 8 | 11 | UP | 86 | GCCAGCCCAG | KRAB-associated protein 1 | Hs.228059 | NM_005762 |
| 1 | 15 | 6 | 6 | UP | 87 | ATGGAAGGTG | hypothetical protein FLJ10702 | Hs.104222 | NM_018184 |
| 1 | 5 | 13 | 8 | UP | 88 | TTTTGTTGCT | hypothetical protein FLJ107I5 | Hs.58974 | NM_018190 |
| 1 | 10 | 5 | 9 | UP | 89 | CCTGAGCCCG | VPS28 | Hs.293441 | BC006485 |
| 1 | 9 | 11 | 6 | UP | 90 | GGTATGCTCC | Similar to presenilins associated rhomboid-like protein | Hs.13094 | BC003653 |
| 1 | 6 | 14 | 5 | UP | 91 | TTTGCTCTCG | vinculin | Hs.75350 | M33308 |
| 1 | 5 | 5 | 9 | UP | 92 | AAACCAGGGC | HSPC166 protein | Hs.279836 | AF161515 |
| 1 | 5 | 7 | 8 | UP | 93 | TGTTTTTATG | TCF1 | Hs.3192 | BC006324 |
| 1 | 6 | 6 | 5 | UP | 94 | TGGAGGCCAG | PR02451 mRNA | Hs.250581 | AF113019 |
| 1 | 8 | 5 | 6 | UP | 95 | TTTTCTGCTG | putative 38.3 kDa protein | Hs.204041 | AF164791 |
| 191 | 30 | 15 | 34 | DN | 96 | ACGCAGGGAG | Histone deacetylase 3 | Hs.279789 | BG779896 |
| 56 | 11 | 3 | 11 | DN | 97 | CCAGAGAACT | PRO1073 protein | Hs.6975 | AF001542 |
| 52 | 0 | 0 | 0 | OFF | 98 | TTGGAACAAT | EST | | BF378356 |
| 50 | 9 | 11 | 5 | DN | 99 | AGGTGGCAAG | EST | | AW275634 |
| 28 | 4 | 0 | 1 | DN | 100 | ATTAAGAGGG | EST | | BG944760 |
| 39 | 0 | 0 | 0 | OFF | 101 | TGCCCTCAGG | Lipocalin 2 | Hs.204238 | BE645920 |
| 25 | 1 | 0 | 2 | DN | 102 | AATGGATGAA | EST | | BG223555 |
| 24 | 1 | 2 | 5 | DN | 103 | GTTGCTCTAT | Tumor protein, translationally-controlled 1 | Hs.279860 AK000037 | |
| 19 | 0 | 0 | 2 | DN | 104 | TCCCTGGCTG | co-beta giucosidase | Hs.78575 | J03077 |
| 18 | 3 | 0 | 3 | DN | 105 | GCAACAACAC | EST | | BG319478 |
| 14 | 1 | 0 | 0 | DN | 106 | CTAACTAGTT | EST | | BE083535 |
| 17 | 0 | 2 | 2 | DN | 107 | GAAGAAGAAG | cDNA DKFZp586G1923 | Hs.28491 | AU099922 |
| 14 | 0 | 0 | 0 | OFF | 108 | GTGTACTTGT | cathepsin S (CTSS) | Hs.181301 | M90696 |
| 13 | 0 | 0 | 0 | OFF | 109 | AACGAGGAAT | EST | | BE694640 |
| 13 | 0 | 0 | 0 | OFF | 110 | GATGATTATC | myeloid cell nuclear differentiation antigen | Hs.153837 | M81750 |
| 12 | 0 | 0 | 1 | DN | 111 | GCCGTTCTTA | EST | | BF813112 |
| 11 | 0 | 0 | 1 | DN | 112 | AGAGGTGTAG | EST | | AA484478 |
| 11 | 0 | 0 | 0 | OFF | 113 | TACTTTTATT | chemokine receptor CXCR4-Lo | Hs.89414 | AF147204 |
| 9 | 1 | 2 | 0 | DN | 114 | GGCAGCACAA | novel heterogeneous nuclear RNP protein | Hs.2730 | X16135 |
| 9 | 1 | 1 | 1 | DN | 115 | GTGCTATTAT | Mcl-1 (MCL-1) and Mcl-1 delta S/TM (MCL-1) genes | Hs.86386 | AF198614 |
| 9 | 0 | 0 | 0 | OFF | 116 | AAGGCTCTCA | EST | | AA377168 |
| 8 | 0 | 1 | 0 | DN | 117 | CTAAAACTTC | EST | Hs.279830 | BE259974 |
| 8 | 0 | 0 | 1 | DN | 118 | AGAATGGTGG | EST | Hs.290340 | BE676676 |
| 7 | 0 | 0 | 1 | DN | 119 | CTAGACGTTG | Rho-associated, coiled-coil containing protein kinase 1 | Hs.17820 | AA593781 |
| 7 | 1 | 0 | 0 | DN | 120 | CTAGCTATTC | EST | Hs.178784 | A1681868 |
| 6 | 0 | 1 | 1 | DN | 121 | CCTTTGGCTA | 40S ribosomal protein S27 isoform | Hs.108957 | BF131624 |
| 6 | 1 | 1 | 0 | DN | 122 | TATGTAAATG | PRO0659 protein | Hs.6451 | NM_014138 |
| 6 | 0 | 0 | 0 | OFF | 123 | AAGCCATTCA | PRO1073 protein | Hs.6975 | BF749571 |
| 6 | 0 | 1 | 0 | DN | 124 | CATTTCTAAA MFGF9 | Hs.5599 | AB011542 | |
| 6 | 1 | 0 | 0 | DN | 125 | ACATTTCATC | Hypothetical protein FLJ23306 | Hs.5890 | BG235945 |
| 6 | 0 | 0 | 1 | DN | 126 | GATACAGTAA | Thrombospondin 1 | Hs.87409 | AW769422 |
| 6 | 0 | 0 | 0 | OFF | 127 | TTATGCTACC | EST | Hs.120770 | A1399644 |
| 6 | 0 | 0 | 0 | OFF | 128 | GATAAAGTGC | Hypthetical protein FLJ22662 | Hs.178470 | BG481449 |
| 5 | 1 | 0 | 1 | DN | 129 | CCGACGGGCG | EST | | BE714831 |
| 5 | 0 | 0 | 0 | OFF | 130 | CCAAACCTAT | Tumor protein, translationally-controlled 1 | Hs.279860 | AW869240 |
| 5 | 0 | 1 | 1 | DN | 131 | TCACCTGAAA | Heterogeneous nuclear ribonucleoprotein A2/B1 | Hs.232400 | W02101 |
| 5 | 1 | 0 | 1 | DN | 132 | ACCTGTGACT | Ribosomal protein S18 | Hs.275865 | AW008211 |
| 5 | 0 | 1 | 0 | DN | 133 | CCCCAGGCTC | KIAA1245 | Hs.218329 | AB033071 |
| 5 | 1 | 0 | 0 | DN | 134 | CCCTTCTGGC | Eukaryotic translation elongation factor 1 alpha 1 | Hs.181165 | BG822114 |
| 5 | 1 | 0 | 1 | DN | 135 | GCACCATAAT | CD164 antigen | Hs.43910 | BF437912 |
| 5 | 1 | 1 | 1 | DN | 136 | TTTTGTGCAG | CD86 antigen | Hs.27954 | AA927298 |
| 5 | 0 | 1 | 1 | DN | 137 | GGCGCTTGTC | EST | Hs.149082 | A1243803 |
| 5 | 0 | 1 | 0 | DN | 138 | AAACCAATTT | FLJ22662 fis, clone RS108080 | Hs.178470 | AK026315 |
| 5 | 0 | 0 | 1 | DN | 139 | AAGGAGAAAG | Methionine-tRNA synthetase | Hs.279946 | AA378028 |
| 5 | 0 | 1 | 0 | DN | 140 | CTGGAACAAA | EST | | BF852658 |
| 5 | 1 | 0 | 0 | DN | 141 | GAGTGACCCT | Hypothetical protein FLJ14950 | Hs.294022 | BE673008 |
| 5 | 0 | 0 | 0 | OFF | 142 | GATGTTTCCA | Diacylglycerol kinase | Hs.115907 | AA921893 |
| 5 | 0 | 0 | 0 | OFF | 143 | TGGCGAGATC | actin dependent regulator of chromatin, subfamily e | Hs.332848 | BG753322 |
| 5 | 0 | 0 | 1 | DN | 144 | TTCAGAATAC | CARD only protein | Hs.348365 | BE019634 |
| 5 | 0 | 0 | 0 | 0FF | 145 | ATACGTAAGT | Upregulated by 1,25-dihydroxyvitamin D-3 | Hs.179526 | S73591 |
| 5 | 0 | 0 | 0 | 0FF | 146 | CTCAACAATG | C-type lectin, superfamily member 12 | Hs.161786 | BG539606 |
| 5 | 0 | 0 | 0 | OFF | 147 | CTTCTTTATA | EST | | AV657195 |
| 5 | 0 | 0 | 0 | 0FF | 148 | GATCGATTTA | helix-loop-helix basic phosphoprotein (G0S8) | Hs.78944 | L1392 |
| 5 | 0 | 0 | 0 | OFF | 149 | GGTTAATGGA | HLA-E Hs.181392 | AA477175 | |
| 5 | 0 | 0 | 0 | 0FF | 150 | GTCTTGGTAG | FLJ23123 fis, clone LNG08039 | Hs.124292 | AK026776 |
| 5 | 0 | 0 | 0 | OFF | 151 | TAAATTAGTC | phospholipase A2 | Hs.75103 | M86400 |
| 5 | 0 | 0 | 0 | OFF | 152 | TTCCAGTTTC | EST | | BG261394 |
| 3 | 19 | 17 | 18 | UP | 153 | GAGAAACCCT | EST | Hs.283487 | A1693217 |
| 0 | 9 | 13 | 15 | ON | 154 | CAGTCTTTTG | cDNA DKFZp434K052 | Hs.169639 | AL137398 |
| 0 | 6 | 6 | 6 | ON | 155 | | EST | | BE894625 |

TABLE 2

Novel 3′ ESTs abnormally expressed in AML with t(9;11)

| | | | | | | | | |
|---|---|---|---|---|---|---|---|---|
| | +HC,5 | AML | | | | | | |
| Normal | M1 | M5 | M5 | SEQ ID NO. | Tag | Expression | SEQ ID NO. | Sequence |

| | | | | | | | | |
|---|---|---|---|---|---|---|---|---|
| 425 | 69 | 92 | 93 | 156 | CTAAGACTTC | DN | 245 | CATGCTAAGACTTCGGCAGTCAAAGCGAACTACTA |
| | | | | | | | | TACTCAATTGATCGAATAAGTTGCCCAACGGAACA |
| | | | | | | | | AGTTACCCTAGGGATAACAGCGCAATCCTATTTTA |
| | | | | | | | | GAGTCCATATCCACAATAGGG |
| 25 | 2 | 1 | 4 | 157 | TCAACTTCTG | DN | 246 | CATGTGAACTTCTGCACACTGGAAGGTGAAACCTG |
| | | | | | | | | GAGAGAGAAGACACTCCCCTCCCTAGCTTCTACCT |
| | | | | | | | | GGCACCCTCCAAAGATGAGCATTCATCTTGGAGA |
| | | | | | | | | CCAAAATAAAAAGGGACAAAAGACCCGGCTCAGA |
| | | | | | | | | GGGAGCAGAGCTCAATGGGGGGAAGTGAAAGCG |
| | | | | | | | | GCCGCTCTAGATAGT |
| 23 | 1 | 1 | 0 | 158 | AGGAAGGAAA | DN | 247 | CATGAGGAAGGAAAGGAAGAAAAGGAAGGAAGG |
| | | | | | | | | AAGAAGGGAAGGAAGGAAAGGAAGGAAAGTAGG |
| | | | | | | | | AAAGGAAGGAAAGG |
| 20 | 0 | 3 | 3 | 159 | AGTAGGTGGC | DN | 248 | CATCAGTAGGTGGCCAGGAAGGGGAAGGCAGGA |
| | | | | | | | | GATACAGAAGG |
| 15 | 2 | 2 | 2 | 160 | CTTTTCTCTT | DN | 249 | CATGGTGGAGGCAGGAGAATAGTGTGAACGTGGG |
| | | | | | | | | AGGCGGAGCTTGCGGTGAGCCAGGATCGCGTCAC |
| | | | | | | | | TGGACTCCAGCCTGGGGGACAGAGTGAGACCCTG |
| | | | | | | | | TCTC |
| 15 | 1 | 0 | 0 | 161 | GTGGAGGCAG | DN | 250 | CATGGTGGAGGCAGGAGACCAAGAAGTTGGGGAG |
| | | | | | | | | AAAAGG |
| 15 | 0 | 0 | 1 | 162 TTACTTATAC | DN | 251 | CATGTTACTTATACTGGATGGTGAAATTGGTTGCT | |
| | | | | | | | | CTTGTATTTTATG |
| 13 | 69 | 55 | 63 | 163 | AACCCGGGAG | UP | 252 | CATGAACCCGGGAGGTGGAGGCTACAGTAAGCTA |
| | | | | | | | | TTATCTCCCCCCTGTACTCCAGCCTGGGTGACAGA |
| | | | | | | | | GCGAGCCCCCAGACTCAAAAAATAAAAATAAAAA |
| | | | | | | | | CCCTGAATATTTTCCTTTT |
| 13 | 1 | 0 | 1 | 164 | CTCATAAGAA | DN | 253 | CATGCTGATAAGAAAGACAGGAAATGGTTTGGCA |
| | | | | | | | | GGTCTTCCAGTGGACATAA |
| 13 | 0 | 0 | 0 | 165 | GCCACACCCA | OFF | 254 | CATGGATGATTATCATTTTACATCAATTAAGTCCT |
| | | | | | | | | TACTGGCCTATGATTTAGGGCTAACTAC |
| 13 | 0 | 0 | 0 | 166 | TGCCCTCAAA | OFF | 255 | CATGTGCCCTCAAA |
| 11 | 0 | 2 | 0 | 167 | ATGCCCACGG | DN | 256 | CATGATGCCCACGGGTACCACTAGAAGCGGAAGC |
| | | | | | | | | GAGAGG |
| 11 | 0 | 1 | 0 | 168 | AATGGAAAAA | DN | 257 | CATGAATGGAAAA |
| 10 | 0 | 0 | 1 | 169 | GGTCAGTCGG | DN | 258 | CATGGGTGAAGTGGGTCCTGAGAGATGGGCGAGC |
| | | | | | | | | GCCGTTCCGAAGGGACGGGCGATGGCCTCCAGCC |
| | | | | | | | | CTTAGAGCCAATCCTTATCCCGAAGTTACGGATCC |
| | | | | | | | | GGCTTGCCGACTTCCCTTACCTACATTGACTCGG |
| 9 | 0 | 0 | 0 | 170 | CATTGCCTTC | OFF | 259 | CATGCATTGCCTTCATTTATTGTATTTCAAATCACT |
| | | | | | | | | GTACATTTACTTTTGTGAAAACACTACCTGCATTT |
| | | | | | | | | TCTAGTAC |
| 8 | 0 | 0 | 0 | 171 | ATCGAAAGGA | OFF | 260 | CATGATCCAAAGGAACAAGCATCAAGCACGCAGC |
| | | | | | | | | AATGCAGCT |
| 8 | 1 | 1 | 0 | 172 | TGTTAATGTT | DN | 261 | CATGTGTTAATGTTGACAATTTAGAAAACACAGAC |
| | | | | | | | | AAGTGAAAGAAAAGAGAAAGAATGAGAAAAGG |
| 8 | 0 | 1 | 0 | 173 | GACAAGAGCG | DN | 262 | CATGGAGAAGAGCGAAACTCCGTCTC |
| 8 | 0 | 0 | 0 | 174 | CCGTCTACAG | OFF | 263 | CATGTGTTAATGTTGAATCAAAACTTTTCTTTAGG |
| | | | | | | | | TGTGCATATAT |
| 8 | 0 | 0 | 0 | 175 | TGCTGCAGAA | OFF | 264 | CATGTCCTGCAGAATAATAAAGTCATCAATACCTC |
| 7 | 0 | 0 | 1 | 176 | ACGCAGGAAG | DN | 265 | GATGACGCAGGAAGCAGCTGAAAGAGCTATTG |
| 7 | 0 | 0 | 0 | 177 | GGACCACGGG | OFF | 266 | CATGGGACCACGGGTGACGGGGAATCAGGGTTCG |
| | | | | | | | | ATTCCCGGAGAGGGAGCCTGAGAAACGGCTACCAC |
| | | | | | | | | ATCCAAGGTAGGCAGCAGGCGCGCAAATTACCCA |
| | | | | | | | | CTGCCGACCCGGGGAGGTAGTGACGAAAAATAAC |
| | | | | | | | | AATACAGGACTCTTTCGAGGCCCTGTAATTGGAAT |
| | | | | | | | | GAGTCCACTTTAAATCCTTTAACGAGGATCCATTG |
| | | | | | | | | GAGGGCAAGTCTGGTGCCAGCAGCCGCGGTAAT |
| | | | | | | | | CCAGCTCCAATAGCG |
| 7 | 1 | 1 | 1 | 178 | TAGTACAGCC | DN | 267 | CATGTAGTALCACGCTCTAAATCACC |
| 7 | 0 | 1 | 0 | 179 | TTCTCATAGG | DN | 268 | CATGTTCTCATAGGAGATCTCCACAGGGGCTGGAC |
| | | | | | | | | GGTTCATTATGGCAAATAAAAAGTGTGAGCGTGC |
| | | | | | | | | GTGTGAGTGTGATGGGGAAAGTG |
| 7 | 0 | 0 | 1 | 180 | GAGAGGTGGA | DN | 269 | CATGGAGAGGTGGAAGACAGAAGTACGGGAAGG |
| | | | | | | | | CGAAGAAAAGAATAGAGAAGATAGGG |
| 7 | 0 | 0 | 0 | 181 | AAGAATGCAG | OFF | 270 | CATGAAGAATGCAGGAAAGAGATCG |
| 7 | 0 | 0 | 0 | 182 | GAGATTATCT | OFF | 271 | CATGGAGATTATCTTTGTTTTAGTTTTCCTTCTAGC |
| | | | | | | | | ATATTATTGTAAAATTAATAGTATATAGAATAACA |
| | | | | | | | | GTTTCTTGCATACTACAGCATTTTTGTCTGATTTTT |
| | | | | | | | | TTTCGTAGTAGCCATAGCC |
| 6 | 0 | 0 | 0 | 183 | ACCCAGGGAG | OFF | 272 | CATGACCCAGGGAGGGTGGTCAGCAGCGCAGCCT |
| | | | | | | | | GCCCAGGCTGGGATCTGGCTTTGGTCATAGCCGTG |
| | | | | | | | | TTTCCATATACGCCTCCACTCACCCTCAGAGGAGG |
| | | | | | | | | AAGGGATGGAAGCCACGAGCATAAATAAAGGGAA |
| | | | | | | | | CACAGAAGAACAATGTCACCAAAGTGCAGGTGCA |
| | | | | | | | | AAGCCCAAAGCAGGCCCCTACCTCTGCCAGCCCA |
| | | | | | | | | GACCCGCCACTAAATTCTAGAGGAGGGTGTCTCTC |
| | | | | | | | | AGGTCACAGTACTT |
| 6 | 1 | 0 | 1 | 184 | TGAGGGAAAA | DN | 273 | CATGTGAGGGAAAATGAAAGCTACTCATAGCGGG |
| | | | | | | | | GGCCT |
| 6 | 0 | 0 | 0 | 185 | AGAAAACAGT | OFF | 274 | CATGAGAAAACAGTATGCCCATTAATTTACTTGT |
| | | | | | | | | GTTCAAAATAAAATTCCAACTGCTGTTGGGGGGG |
| | | | | | | | | GCCCCAGAAACGAAAAATAACCCCGCC |
| 6 | 1 | 0 | 1 | 186 | CTCATAAAGA | DN | 275 | CATGCTCATAAAGA |
| 6 | 1 | 1 | 0 | 187 | TATGAAAACA | DN | 276 | CATGTATGAAAACAGAAGACAAAATTGTGAGCCA |
| | | | | | | | | GAGTCGACAAATGAAATAAATTACCCCCTCCTCCG |
| 6 | 1 | 0 | 0 | 188 | AGGTTGACAG | DN | 277 | CATGAGGTTGACAGTGTAAACCTGCTCTAAGTTTT |
| | | | | | | | | CACTTTGCACTGTGTACCAAAATGAAACTGCTTAT |
| | | | | | | | | TTAGGAAAATAAAAATATCTTTTGTGTTGGAC |
| 6 | 0 | 0 | 0 | 189 | CTTCCTTCCA | OFF | 278 | CATGAGGTTGACAGTGAGGGTAATAAAAGTAAAA |
| | | | | | | | | GGAACTAATTTTGGAAAAGCTAAGACAGAAGAAA |
| | | | | | | | | AGGACCTTAAGAAGCTAAAAAAGCAGG |
| 6 | 0 | 1 | 0 | 190 | GTAACTGATT | DN | 279 | CATGGTAACTGATTCTTAACAGATTCATATATGTA |
| | | | | | | | | TCTGT |
| 6 | 0 | 0 | 0 | 191 | TTAATTACAG | OFF | 280 | CATGTTAATTACAGCACATTAAAATGGTGGTTTAC |
| | | | | | | | | ATTACAAATAAGCCTGTAAGTTTAAATATACTAGT |
| | | | | | | | | GTTATAACCCAATGTACAGACGTTCTTTATACAAT |
| | | | | | | | | ACATACAATTATCAGGAATGC |
| 6 | 0 | 0 | 0 | 192 | GAGGAGGATT | OFF | 281 | CATGGAGGACCATTAAAATGTGACACCTTTC |
| 6 | 0 | 0 | 0 | 193 | TGCCCTCAGA | OFF | 282 | CATGTGCCCTCAGAG |
| 5 | 0 | 0 | 0 | 194 | GAGATCACAA | OFF | 283 | CATGTGCCCTCAGATTCCGCACCAATAAAGCCTTC |
| | | | | | | | | AAACTCCCG |
| 5 | 1 | 1 | 1 | 195 | TAAGTGGAAT | DN | 284 | CATGTAAAGTGGAATAAAAGTTTTATGAATGGAC |
| | | | | | | | | TTTT |
| 5 | 1 | 1 | 1 | 196 | CTTAATCAGT DN | 285 | CATGCTTAATCACTGCAATTTTAAGCTACTGTACA | |
| | | | | | | | | CAGGAATGAAAAGGTTATAGAAAAGTGCCATAGC |
| | | | | | | | | AACAGTGCCTTAAGAAAGGAGATAAAGAGGAGCC |
| | | | | | | | | TTAAAAAAATGGATAAAATCAGAATTTCAGAAGG |
| | | | | | | | | AAATGGAAACACACGGGAAATGAAAAACATTTCT |
| | | | | | | | | CTGC |
| 5 | 0 | 1 | 1 | 197 | ATGGCTCTTG | DN | 286 | CATGATGGCTCTTGATGGCAGTTAGCTCTTATTGC |
| | | | | | | | | TGAAAATAAAATAAAAATAAATAAAAAAGCGGCC |
| | | | | | | | | GCTCTAGATAG |
| 5 | 1 | 0 | 0 | 198 | GAGCACTTGG | DN | 287 | CATGGAGCACTTGGCGTGCCATAATGATGGGAAT |
| | | | | | | | | AGCTGTCAGCTGTAAGATCACCATTGCTGACTGGA |
| | | | | | | | | CAACTGCAATAAATTTGACGGGTGTTTCTCTT |
| 5 | 1 | 1 | 0 | 199 | GTTCAGGAAA | DN | 288 | CATGGTTCAGGAAA |
| 5 | 1 | 1 | 0 | 200 | TGGCCCAGTA | DN | 289 | CATGTGGCCCAGTATGTGGTACGGG |
| 5 | 0 | 1 | 1 | 201 | GACAGTAGAT | DN | 290 | CATGGACAGTAGATGG |
| 5 | 1 | 1 | 0 | 202 | AGGGAGGCAG | DN | 291 | CATGAGGGAGGCAGAGGTTGCAGTGAGCCGAGAT |
| | | | | | | | | CGAACCATCGCATTCCAGCCTTGGCGACAGAATG |
| | | | | | | | | AGACACTGTTTC |
| 5 | 0 | 0 | 0 | 203 | AGTGAGAAGG | OFF | 292 | CATGAGTGAGAAGGCAGGTTGTGCGGGTGTTGAC |
| | | | | | | | | CGATGTATCTTTTCCTTAAAGTTATTATAATAATG |
| | | | | | | | | CCTCCGTAATTTGTCAATAAAGCATTCCTTTGGGG |
| 5 | 0 | 1 | 1 | 204 | GGCCTGGCAC | DN | 293 | CATGGGCCTGGCACTGCACACTCAGTTCTGCTCTA |
| | | | | | | | | AGAAGCTGCAATAAAGTTTTTTTAAGTCACTTTGG |
| 5 | 0 | 1 | 1 | 205 | GTTAAATGCA | DN | 294 | CATGGTTAAATGCATTTCCTAATTTGAGATCCCCT |
| | | | | | | | | AAACCCTGG |
| 5 | 0 | 1 | 1 | 206 | TGAAAGTCTT | DN | 295 | CATGTGAAAGTTTTATGCTCTGCATTTGCCCCCTGG |
| | | | | | | | | TGATTTTAAAATTTGTTTGCCTTTTTAAAGCTATAT |
| | | | | | | | | TAAAAATGTATTGTTGAATC |
| 5 | 0 | 0 | 0 | 207 | ATCTTACCTG | OFF | 296 | CATGATCTTACCTGCTCGAATTCAAGCTTGTAACG |
| | | | | | | | | ATGTACGGGAGATGGCCGTTCTTAGTTGGTGGAGC |
| | | | | | | | | GATTTGTCTGGTTAATTCCGATAACG |
| 5 | 1 | 0 | 0 | 208 | CATTTGTCTT | DN | 297 | CATGCATTTGTCTTTTCATAAAAAAATTACGTAAA |
| | | | | | | | | AATGCCCCCAGG |
| 5 | 0 | 0 | 1 | 209 | GTGCACTACC | DN | 298 | CATGCTGCACTACCAGGTTCTAGGAAGAGGAGAA |
| | | | | | | | | GAAAGGAGGAAACCACAGGG |
| 5 | 0 | 1 | 0 | 210 | TATAACAAAA | DN | 299 | CATGTATAACAAAAGCTTATTTCATTATTATTAT |
| | | | | | | | | AATAAACAGTTAATGGTTGAGACATTTT |
| 5 | 0 | 0 | 0 | 211 | ACAGCAACCT | OFF | 300 | CATGACAGCAACCTGGATGGAACTGGAGGCTGTT |
| | | | | | | | | ATCTTAAGTGAAATAACTTAGAAGTAGAAAGTC |
| 5 | 0 | 0 | 0 | 212 | ACCTGCAGAA | OFF | 301 | CATGACCTGCAGAATAATAAAGTCATCAATACCG |
| 5 | 0 | 0 | 0 | 213 | CGACGACGCT | OFF | 302 | CATGCGACGACGCTATGAACGCTTGGCCGCCACA |
| | | | | | | | | AGCCAGTTATCCCTGTGGTAACTTTTCTGACACCT |
| | | | | | | | | CGTGCTTAAAACCC |
| 5 | 0 | 0 | 0 | 214 | CTCAAAAGGA | OFF | 303 | CATGGTCAAAAGGA |
| 5 | 0 | 0 | 0 | 215 | TAAGAGGACA | OFF | 304 | CATGTAAGAGGACATGAGGAAGG |
| 5 | 0 | 0 | 0 | 216 | TGACGATCAT | OFF | 305 | CATGTGACCATCATAGCCAGCATCACCCTCCTTAA |
| | | | | | | | | CCTCTACTTCTACCTACGCGTAATCTACTCCACGTC |
| | | | | | | | | AATCACACTACCCCCATATCTAACAACGT |
| 5 | 0 | 0 | 0 | 217 | TTACTTGTCG | OFF | 306 | CATGTTACTTGTCG |
| 2 | 24 | 20 | 16 | 218 | CTTTTTGTGC | UP | 307 | CATGTTTTTGTGCTCCCCTTTGCCTAAACCGTATG |
| | | | | | | | | GCCTCCCGTGCATCTGTACTCACCCTGTACGACAA |
| | | | | | | | | ACACATTACATTATTAAATGTTTCTCAAAGATGG |
| 2 | 11 | 11 | 20 | 219 | TGCTGGGTGG | UP | 308 | CATGTGCTGGGTGGGTTCAGAGGGCAATTTCTCTT |
| | | | | | | | TTATGTGTAGATATGCTAAATAAACATAATTT | |
| 2 | 9 | 17 | 9 | 220 | CCTGTAGTTC | UP | 309 | CATGCCTGTAGTTCCAGTTACTGGAGTGGAGTGGC |
| | | | | | | | | TGAGTTAGGAAGACAGGAGGATAGCTTGATTCTA |
| | | | | | | | | AGAGGTTGAGTCTAGCCTGGACAATATAGTGAGA |
| | | | | | | | | TGCTGTCTGT |
| 1 | 186 | 68 | 25 | 221 | AAGCGGCCG | ON | 310 | CATGAAAGCGGCCGCTCTAGATAGT |
| 1 | 6 | 7 | 9 | 222 | TACCCGCCTC | UP | 311 | CATCTACCCGCCTCCTTTCTGTTTTATTTTTGAGGA |
| | | | | | | | | AATAAAATAACCAAGTGCT |
| 0 | 6 | 6 | 5 | 223 | GTGGTGCGCG | ON | 312 | CATGACACAGCAAGCGGCCGCTCTAGATAG |

TABLE 3

Genes abnormally expressed in t(9;11)
leukemic cells

| | | | | | | |
|---|---|---|---|---|---|---|
| | | AML | | | | |
| Normal | M1 | M5 | M5 | Expression | SEQ ID NO | SAGE Tag |

| | | | | | | |
|---|---|---|---|---|---|---|
| 102 | 11 | 13 | 12 | DN | 224 | ACTTTCCAAA |
| 10 | 0 | 0 | 0 | OFF | 225 | GTGACCCCGG |
| 10 | 0 | 0 | 0 | OFF | 226 | GCGACCACGG |
| 9 | 1 | 2 | 0 | DN | 227 | CGTGGAAGCA |
| 8 | 0 | 0 | 0 | OFF | 228 | GGGACCACGG |
| 7 | 1 | 0 | 0 | DN | 229 | GTGACCAGGG |
| 7 | 0 | 0 | 1 | DN | 230 | GAAGTCGGAA |
| 7 | 0 | 0 | 0 | OFF | 231 | CCACGGCCCT |
| 7 | 0 | 0 | 0 | OFF | 232 | GCAGCAAAAG |
| 6 | 1 | 0 | 0 | DN | 233 | CAAGTGGCAA |
| 6 | 1 | 1 | 1 | DN | 234 | TGTAATCTTA |
| 6 | 1 | 0 | 0 | DN | 235 | GCAGCTCCAT |
| 5 | 0 | 0 | 0 | OFF | 236 | GTGACACGGG |
| 5 | 1 | 1 | 0 | DN | 237 | CTCTTCTCTC |
| 5 | 0 | 0 | 0 | OFF | 238 | CTTCTTGCCC |
| 2 | 49 | 12 | 22 | UP | 239 | TACTCTGGGT |
| 1 | 133 | 32 | 24 | ON | 240 | AAGCGGCCGC |
| 1 | 12 | 6 | 16 | UP | 241 | CACTTAATTG |
| 1 | 5 | 11 | 8 | UP | 242 | TCCCCGTACA |
| 1 | 5 | 6 | 6 | UP | 243 | GAGAGCTCCC |
| 0 | 6 | 8 | 10 | ON | 244 | ACACAGCAAG |

# SEQUENCE LISTING
<160> NUMBER OF SEQ ID NOS: 315
<210> SEQ ID NO 1
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 1
ctgacttgtg
#
#
# 10
<210> SEQ ID NO 2
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 2
actttaatga
#
#
# 10
<210> SEQ ID NO 3
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 3
gagtaaaaaa
#
#
# 10
<210> SEQ ID NO 4
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 4
cttttttccc
#
#
# 10
<210> SEQ ID NO 5
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 5
gctccccttt
#
#
# 10
<210> SEQ ID NO 6
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 6
ggtacccatt
#
#
# 10
<210> SEQ ID NO 7
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 7
ctgagaaact
#
#
# 10
<210> SEQ ID NO 8
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 8
gtgtgtttgt
#
#
# 10
<210> SEQ ID NO 9
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 9
gtcctagcac
#
#
# 10
<210> SEQ ID NO 10
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 10
ctgtacatac
#
#
# 10
<210> SEQ ID NO 11
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 11
tcaccttagg
#
#
# 10
<210> SEQ ID NO 12
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 12
ggccaggact
#
#
# 10
<210> SEQ ID NO 13
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 13
aggtcttcaa
#
#
# 10
<210> SEQ ID NO 14
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 14
agcctcggcc
#
#
# 10
<210> SEQ ID NO 15
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 15
caatttgtgt
#
#
# 10
<210> SEQ ID NO 16
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 16
agttatttct
#
#
# 10
<210> SEQ ID NO 17
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 17
ctaatattgt
#
#
# 10
<210> SEQ ID NO 18
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 18
gaggggatgt
#
#
# 10
<210> SEQ ID NO 19
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 19
ttattgggta
#
#
# 10
<210> SEQ ID NO 20
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 20
ggctttgggg
#
#
# 10
<210> SEQ ID NO 21
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 21
cttttcaaga
#
#
# 10
<210> SEQ ID NO 22
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 22
tgtccacaca
#
#
# 10
<210> SEQ ID NO 23
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 23
acttcacaaa
#
#
# 10
<210> SEQ ID NO 24
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 24
gaacgtctta
#
#
# 10
<210> SEQ ID NO 25
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 25
caagcaaaat
#
#
# 10
<210> SEQ ID NO 26
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 26
aagatgcaca
#
#
# 10
<210> SEQ ID NO 27
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 27
tcagatggcg
#
#
# 10
<210> SEQ ID NO 28
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 28
ctcaaccccc
#
#
# 10
<210> SEQ ID NO 29
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 29
gactggaccg
#
#
# 10
<210> SEQ ID NO 30
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 30
tggtcatact
#
#
# 10
<210> SEQ ID NO 31
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 31
aggacagaag
#
#
# 10
<210> SEQ ID NO 32
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 32
agaaataaag
#
#
# 10
<210> SEQ ID NO 33
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 33
caccacggtg
#
#
# 10
<210> SEQ ID NO 34
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 34
cctggagcaa
#
#
# 10
<210> SEQ ID NO 35
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 35
ggaaggcagt
#
#
# 10
<210> SEQ ID NO 36
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 36
gggcacaatg
#
#
# 10
<210> SEQ ID NO 37
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 37
tatatataca
#
#
# 10
<210> SEQ ID NO 38
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 38
agataatcag
#
#
# 10
<210> SEQ ID NO 39
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 39
cattaaagta
#
#
# 10
<210> SEQ ID NO 40
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 40
cccacactac
#
#
# 10
<210> SEQ ID NO 41
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 41
gatgttattg
#
#
# 10
<210> SEQ ID NO 42
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 42
tacacacgga
#
#
# 10
<210> SEQ ID NO 43
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 43
aatcaatgtc
#
#
# 10
<210> SEQ ID NO 44
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 44
acggtccagg
#
#
# 10
<210> SEQ ID NO 45
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 45
ggctcatctt
#
#
# 10
<210> SEQ ID NO 46
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 46
tccttaggct
#
#
# 10
<210> SEQ ID NO 47
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 47
ggaaaagtgg
#
#
# 10
<210> SEQ ID NO 48
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 48
gccaaaaacc
#
#
# 10
<210> SEQ ID NO 49
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 49
gttataatac
#
#
# 10
<210> SEQ ID NO 50
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 50
acatttccaa
#
#
# 10
<210> SEQ ID NO 51
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 51
atcttgaaag
#
#
# 10
<210> SEQ ID NO 52
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 52
tgagccaaat
#
#
# 10
<210> SEQ ID NO 53
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 53
ctgaagctaa
#
#
# 10
<210> SEQ ID NO 54
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 54
gcaaaacaac
#
#
# 10
<210> SEQ ID NO 55
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 55
gtgaccacag
#
#
# 10
<210> SEQ ID NO 56
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 56
gaactggaga
#
#
# 10
<210> SEQ ID NO 57
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 57
gcctgctatt
#
#
# 10
<210> SEQ ID NO 58
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 58
ccagcggctg
#
#
# 10
<210> SEQ ID NO 59
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 59
tatctgtata
#
#
# 10
<210> SEQ ID NO 60
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 60
aaacgctact
#
#
# 10
<210> SEQ ID NO 61
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 61
atgagaccct
#
#
# 10
<210> SEQ ID NO 62
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 62
ggaagagtgc
#
#
# 10
<210> SEQ ID NO 63
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 63
tagtttgaag
#
#
# 10
<210> SEQ ID NO 64
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 64
tatgaggagg
#
#
# 10
<210> SEQ ID NO 65
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 65
gaaggtgatc
#
#
# 10
<210> SEQ ID NO 66
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 66
ggcagaggac
#
#
# 10
<210> SEQ ID NO 67
<211> LENGTH: 10
<212> TYPE: DNA
<213> ORGANISM: Artificial Sequence
<220> FEATURE:
<223> OTHER INFORMATION: Synthetic oligonucleotide
<400> SEQUENCE: 67
cagtcttttg
#
#
# 10
<210> SEQ ID NO 68
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 68
tgaaagtgtg
#
#
# 10
<210> SEQ ID NO 69
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 69
gaaatccgca
#
#
# 10
<210> SEQ ID NO 70
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 70
ccaccccgaa
#
#
# 10
<210> SEQ ID NO 71
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 71
tttaaaactt
#
#
# 10
<210> SEQ ID NO 72
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 72
ccactctggc
#
#
# 10
<210> SEQ ID NO 73
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 73
aggtcctagc
#
#
# 10
<210> SEQ ID NO 74
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 74
aacctgggag
#
#
# 10
<210> SEQ ID NO 75
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 75
ggaagagctg
#
#
# 10
<210> SEQ ID NO 76
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 76
agaattgctt
#
#
# 10
<210> SEQ ID NO 77
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 77
catatcatta
#
#
# 10
<210> SEQ ID NO 78
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 78
taattcttct
#
#
# 10
<210> SEQ ID NO 79
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 79
aagatccccg
#
#
# 10
<210> SEQ ID NO 80
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 80
aattttattt
#
#
# 10
<210> SEQ ID NO 81
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 81
cacagagtcc
#
#
# 10
<210> SEQ ID NO 82
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 82
tggttgattt
#
#
# 10
<210> SEQ ID NO 83
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 83
acccttccct
#
#
# 10
<210> SEQ ID NO 84
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 84
gttacaaact
#
#
# 10
<210> SEQ ID NO 85
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 85
ctttgatgtt
#
#
# 10
<210> SEQ ID NO 86
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 86
gccagcccag
#
#
# 10
<210> SEQ ID NO 87
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 87
atggaaggtg
#
#
# 10
<210> SEQ ID NO 88
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 88
ttttgttgct
#
#
# 10
<210> SEQ ID NO 89
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 89
cctgagcccg
#
#
# 10
<210> SEQ ID NO 90
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 90
gctatgctcc
#
#
# 10
<210> SEQ ID NO 91
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 91
tttgctctcc
#
#
# 10
<210> SEQ ID NO 92
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 92
aaaccagggc
#
#
# 10
<210> SEQ ID NO 93
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 93
tgtttttatg
#
#
# 10
<210> SEQ ID NO 94
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 94
tggaggccag
#
#
# 10
<210> SEQ ID NO 95
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 95
ttttctgctg
#
#
# 10
<210> SEQ ID NO 96
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 96
acgcagggag
#
#
# 10
<210> SEQ ID NO 97
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 97
ccagagaact
#
#
# 10
<210> SEQ ID NO 98
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 98
ttggaacaat
#
#
# 10
<210> SEQ ID NO 99
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 99
aggtggcaag
#
#
# 10
<210> SEQ ID NO 100
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 100
attaagaggg
#
#
# 10
<210> SEQ ID NO 101
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 101
tgccctcagg
#
#
# 10
<210> SEQ ID NO 102
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 102
aatggatgaa
#
#
# 10
<210> SEQ ID NO 103
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 103
gttgctctat
#
#
# 10
<210> SEQ ID NO 104
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 104
tccctggctg
#
#
# 10
<210> SEQ ID NO 105
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 105
gcaacaacac
#
#
# 10
<210> SEQ ID NO 106
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 106
ctaactagtt
#
#
# 10
<210> SEQ ID NO 107
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 107
gaagaacaag
#
#
# 10
<210> SEQ ID NO 108
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 108
gtgtacttgt
#
#
# 10
<210> SEQ ID NO 109
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 109
aacgaggaat
#
#
# 10
<210> SEQ ID NO 110
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 110
gatgattatc
#
#
# 10
<210> SEQ ID NO 111
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 111
gccgttctta
#
#
# 10
<210> SEQ ID NO 112
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 112
agaggtgtag
#
#
# 10
<210> SEQ ID NO 113
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 113
tacttttatt
#
#
# 10
<210> SEQ ID NO 114
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 114
ggcagcacaa
#
#
# 10
<210> SEQ ID NO 115
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 115
gtgctattat
#
#
# 10
<210> SEQ ID NO 116
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 116
aaggctctca
#
#
# 10
<210> SEQ ID NO 117
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 117
ctaaaacttc
#
#
# 10
<210> SEQ ID NO 118
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 118
agaatggtgg
#
#
# 10
<210> SEQ ID NO 119
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 119
ctagacgttg
#
#
# 10
<210> SEQ ID NO 120
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 120
ctagctattc
#
#
# 10
<210> SEQ ID NO 121
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 121
cctttggcta
#
#
# 10
<210> SEQ ID NO 122
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 122
tatgtaaatg
#
#
# 10
<210> SEQ ID NO 123
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 123
aagccattca
#
#
# 10
<210> SEQ ID NO 124
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 124
catttctaaa
#
#
# 10
<210> SEQ ID NO 125
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 125
acatttcatc
#
#
# 10
<210> SEQ ID NO 126
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 126
gatacagtaa
#
#
# 10
<210> SEQ ID NO 127
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 127
ttatgctacc
#
#
# 10
<210> SEQ ID NO 128
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 128
gataaagtgc
#
#
# 10
<210> SEQ ID NO 129
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 129
ccgacgggcg
#
#
# 10
<210> SEQ ID NO 130
<211> LENGTH: 10
<212> TYPE: DNA
<213> ORGANISM: Artificial sequence
<220> FEATURE:
<223> OTHER INFORMATION: Synhtetic oligonucleotide
<400> SEQUENCE: 130
ccaaacctat
#
#
# 10
<210> SEQ ID NO 131
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 131
tcacctgaaa
#
#
# 10
<210> SEQ ID NO 132
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 132
acctgtgact
#
#
# 10
<210> SEQ ID NO 133
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 133
ccccaggctc
#
#
# 10
<210> SEQ ID NO 134
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 134
cccttctggc
#
#
# 10
<210> SEQ ID NO 135
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 135
gcaccataat
#
#
# 10
<210> SEQ ID NO 136
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 136
ttttgtgcag
#
#
# 10
<210> SEQ ID NO 137
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 137
ggcgcttgtc
#
#
# 10
<210> SEQ ID NO 138
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 138
aaaccaattt
#
#
# 10
<210> SEQ ID NO 139
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 139
aaggagaaag
#
#
# 10
<210> SEQ ID NO 140
<211> LENGTH: 10
<212> TYPE: DNA
<213> ORGANISM: Artificial sequence
<220> FEATURE:
<223> OTHER INFORMATION: Sybnthetic oligonucleotide
<400> SEQUENCE: 140
ctggaacaaa
#
#
# 10
<210> SEQ ID NO 141
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 141
gagtgaccct
#
#
# 10
<210> SEQ ID NO 142
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 142
gatgtttcca
#
#
# 10
<210> SEQ ID NO 143
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 143
tggcgagatc
#
#
# 10
<210> SEQ ID NO 144
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 144
ttcagaatac
#
#
# 10
<210> SEQ ID NO 145
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 145
atacgtaagt
#
#
# 10
<210> SEQ ID NO 146
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 146
ctcaacaatg
#
#
# 10
<210> SEQ ID NO 147
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 147
cttctttata
#
#
# 10
<210> SEQ ID NO 148
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 148
gatcgattta
#
#
# 10
<210> SEQ ID NO 149
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 149
ggttaatgga
#
#
# 10
<210> SEQ ID NO 150
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 150
gtcttggtag
#
#
# 10
<210> SEQ ID NO 151
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 151
taaattagtc
#
#
# 10
<210> SEQ ID NO 152
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 152
ttccagtttc
#
#
# 10
<210> SEQ ID NO 153
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 153
gagaaaccct
#
#
# 10
<210> SEQ ID NO 154
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 154
cagtcttttg
#
#
# 10
<210> SEQ ID NO 155
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 155
tggacccccc
#
#
# 10
<210> SEQ ID NO 156
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 156
ctaagacttc
#
#
# 10
<210> SEQ ID NO 157
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 157
tcaacttctg
#
#
# 10
<210> SEQ ID NO 158
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 158
aggaaggaaa
#
#
# 10
<210> SEQ ID NO 159
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 159
agtaggtggc
#
#
# 10
<210> SEQ ID NO 160
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 160
cttttctctt
#
#
# 10
<210> SEQ ID NO 161
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 161
gtggaggcag
#
#
# 10
<210> SEQ ID NO 162
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 162
ttacttatac
#
#
# 10
<210> SEQ ID NO 163
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 163
aacccgggag
#
#
# 10
<210> SEQ ID NO 164
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 164
ctcataagaa
#
#
# 10
<210> SEQ ID NO 165
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 165
gccacaccca
#
#
# 10
<210> SEQ ID NO 166
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 166
tgccctcaaa
#
#
# 10
<210> SEQ ID NO 167
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 167
atgcccacgg
#
#
# 10
<210> SEQ ID NO 168
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 168
aatggaaaaa
#
#
# 10
<210> SEQ ID NO 169
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 169
ggtcagtcgg
#
#
# 10
<210> SEQ ID NO 170
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 170
cattgccttc
#
#
# 10
<210> SEQ ID NO 171
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 171
atccaaagga
#
#
# 10
<210> SEQ ID NO 172
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 172
tgttaatgtt
#
#
# 10
<210> SEQ ID NO 173
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 173
gacaagagcg
#
#
# 10
<210> SEQ ID NO 174
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 174
ccgtctacag
#
#
# 10
<210> SEQ ID NO 175
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 175
tcctgcagaa
#
#
# 10
<210> SEQ ID NO 176
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 176
acgcaggaag
#
#
# 10
<210> SEQ ID NO 177
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 177
ggaccacggg
#
#
# 10
<210> SEQ ID NO 178
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 178
tagtacaccc
#
#
# 10
<210> SEQ ID NO 179
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 179
ttctcatagg
#
#
# 10
<210> SEQ ID NO 180
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 180
gagaggtgga
#
#
# 10
<210> SEQ ID NO 181
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 181
aagaatgcag
#
#
# 10
<210> SEQ ID NO 182
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 182
gagattatct
#
#
# 10
<210> SEQ ID NO 183
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 183
acccagggag
#
#
# 10
<210> SEQ ID NO 184
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 184
tgagggaaaa
#
#
# 10
<210> SEQ ID NO 185
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 185
agaaaacagt
#
#
# 10
<210> SEQ ID NO 186
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 186
ctcataaaga
#
#
# 10
<210> SEQ ID NO 187
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 187
tatgaaaaca
#
#
# 10
<210> SEQ ID NO 188
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 188
aggttgacag
#
#
# 10
<210> SEQ ID NO 189
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 189
cttccttcca
#
#
# 10
<210> SEQ ID NO 190
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 190
gtaactgatt
#
#
# 10
<210> SEQ ID NO 191
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 191
ttaattacag
#
#
# 10
<210> SEQ ID NO 192
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 192
gaggaccatt
#
#
# 10
<210> SEQ ID NO 193
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 193
tgccctcaga
#
#
# 10
<210> SEQ ID NO 194
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 194
gagatcacaa
#
#
# 10
<210> SEQ ID NO 195
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 195
taagtggaat
#
#
# 10
<210> SEQ ID NO 196
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 196
cttaatcact
#
#
# 10
<210> SEQ ID NO 197
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 197
atggctcttg
#
#
# 10
<210> SEQ ID NO 198
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 198
gagcacttgg
#
#
# 10
<210> SEQ ID NO 199
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 199
gttcaggaaa
#
#
# 10
<210> SEQ ID NO 200
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 200
tggcccagta
#
#
# 10
<210> SEQ ID NO 201
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 201
gacagtagat
#
#
# 10
<210> SEQ ID NO 202
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 202
agggaggcag
#
#
# 10
<210> SEQ ID NO 203
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 203
agtgagaagg
#
#
# 10
<210> SEQ ID NO 204
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 204
ggcctggcac
#
#
# 10
<210> SEQ ID NO 205
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 205
gttaaatgca
#
#
# 10
<210> SEQ ID NO 206
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 206
tgaaagtctt
#
#
# 10
<210> SEQ ID NO 207
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 207
atcttacctg
#
#
# 10
<210> SEQ ID NO 208
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 208
catttgtctt
#
#
# 10
<210> SEQ ID NO 209
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 209
ctgcactacc
#
#
# 10
<210> SEQ ID NO 210
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 210
tataacaaaa
#
#
# 10
<210> SEQ ID NO 211
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 211
acagcaacct
#
#
# 10
<210> SEQ ID NO 212
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 212
acctgcagaa
#
#
# 10
<210> SEQ ID NO 213
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 213
cgacgacgct
#
#
# 10
<210> SEQ ID NO 214
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 214
ctcaaaagga
#
#
# 10
<210> SEQ ID NO 215
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 215
taagaggaca
#
#
# 10
<210> SEQ ID NO 216
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 216
tgaccatcat
#
#
# 10
<210> SEQ ID NO 217
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 217
ttacttgtcg
#
#
# 10
<210> SEQ ID NO 218
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 218
ctttttgtgc
#
#
# 10
<210> SEQ ID NO 219
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 219
tgctgggtgg
#
#
# 10
<210> SEQ ID NO 220
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 220
cctgtagttc
#
#
# 10
<210> SEQ ID NO 221
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 221
aaagcggccg
#
#
# 10
<210> SEQ ID NO 222
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 222
tacccgcctc
#
#
# 10
<210> SEQ ID NO 223
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 223
gtggtgcgcg
#
#
# 10
<210> SEQ ID NO 224
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 224
actttccaaa
#
#
# 10
<210> SEQ ID NO 225
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 225
gtgaccccgg
#
#
# 10
<210> SEQ ID NO 226
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 226
gcgaccacgg
#
#
# 10
<210> SEQ ID NO 227
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 227
cgtggaagca
#
#
# 10
<210> SEQ ID NO 228
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 228
gggaccacgg
#
#
# 10
<210> SEQ ID NO 229
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 229
gtgaccaggg
#
#
# 10
<210> SEQ ID NO 230
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 230
gaagtcggaa
#
#
# 10
<210> SEQ ID NO 231
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 231
ccacggccct
#
#
# 10
<210> SEQ ID NO 232
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 232
gcagcaaaag
#
#
# 10
<210> SEQ ID NO 233
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 233
caagtggcaa
#
#
# 10
<210> SEQ ID NO 234
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 234
tgtaatctta
#
#
# 10
<210> SEQ ID NO 235
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 235
gcagctccat
#
#
# 10
<210> SEQ ID NO 236
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 236
gtgacacggg
#
#
# 10
<210> SEQ ID NO 237
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 237
ctcttctctc
#
#
# 10
<210> SEQ ID NO 238
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 238
cttcttgccc
#
#
# 10
<210> SEQ ID NO 239
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 239
tactctgggt
#
#
# 10
<210> SEQ ID NO 240
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 240
aagcggccgc
#
#
# 10
<210> SEQ ID NO 241
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 241
cacttaattg
#
#
# 10
<210> SEQ ID NO 242
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 242
tccccgtaca
#
#
# 10
<210> SEQ ID NO 243
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 243
gagagctccc
#
#
# 10
<210> SEQ ID NO 244
<211> LENGTH: 10
<212> TYPE: DNA
<213> ORGANISM: Artificial sequence
<220> FEATURE:
<223> OTHER INFORMATION: Synthetic oligonucleotide
<400> SEQUENCE: 244
acacagcaag
#
#
# 10
<210> SEQ ID NO 245
<211> LENGTH: 126
<212> TYPE: DNA
<213> ORGANISM: Artificial sequence
<220> FEATURE:
<223> OTHER INFORMATION: Synthetic oligonucleotide
<400> SEQUENCE: 245
catgctaaga cttccccagt caaagcgaac tactatactc aattgatcca at
#aacttgcc 60
caacggaaca agttacccta gggataacag cgcaatccta ttttagagtc ca
#tatccaca 120
ataggg
#
#
# 126
<210> SEQ ID NO 246
<211> LENGTH: 186
<212> TYPE: DNA
<213> ORGANISM: Artificial sequence
<220> FEATURE:
<223> OTHER INFORMATION: Synthetic oligonucleotide
<400> SEQUENCE: 246
catgtcaact tctgcacact ggaaggtgaa acctggagag agaagacact cc
#cctcccta 60
gcttctacct ggcaccctcc aaagatgagc attcatcttg gagaccaaaa ta
#aaaaggga 120
caaaagaccc ggctcagagg gagcagagct caatgggggg aagtgaaagc gg
#ccgctcta 180
gatagt
#
#
# 186
<210> SEQ ID NO 247
<211> LENGTH: 80
<212> TYPE: DNA
<213> ORGANISM: Artificial sequence
<220> FEATURE:
<223> OTHER INFORMATION: Synthetic oligonucleotide
<400> SEQUENCE: 247
catgaggaag gaaaggaaga aaaggaagga aggaagaagg gaaggaagga aa
#ggaaggaa 60
agtaggaaag gaaggaaagg
#
#
# 80
<210> SEQ ID NO 248
<211> LENGTH: 44
<212> TYPE: DNA
<213> ORGANISM: Artificial sequence
<220> FEATURE:
<223> OTHER INFORMATION: Synthetic oligonucleotide
<400> SEQUENCE: 248
catgagtagg tggccaggaa ggggaaggca ggagatacag aagg
#
# 44
<210> SEQ ID NO 249
<211> LENGTH: 106
<212> TYPE: DNA
<213> ORGANISM: Artificial sequence
<220> FEATURE:
<223> OTHER INFORMATION: Synthetic oligonucleotide
<400> SEQUENCE: 249
catggtggag gcaggagaat agtgtgaacg tgggaggcgg agcttgcggt ga
#gccaggat 60
cgcgtcactg cactccagcc tgggcgacag agtgagaccc tgtctc
# 106
<210> SEQ ID NO 250
<211> LENGTH: 40
<212> TYPE: DNA
<213> ORGANISM: Artificial sequence
<220> FEATURE:
<223> OTHER INFORMATION: Synthetic oligonucleotide
<400> SEQUENCE: 250
catggtggag gcaggagacc aagaagttgg ggagaaaagg
#
# 40
<210> SEQ ID NO 251
<211> LENGTH: 48
<212> TYPE: DNA
<213> ORGANISM: Artificial sequence
<220> FEATURE:
<223> OTHER INFORMATION: Synthetic oligonucleotide
<400> SEQUENCE: 251
catgttactt atactggatg gtgaaattgg ttgctcttgt attttatg
# 48
<210> SEQ ID NO 252
<211> LENGTH: 122
<212> TYPE: DNA
<213> ORGANISM: Artificial sequence
<220> FEATURE:
<223> OTHER INFORMATION: Synthetic oligonucleotide
<400> SEQUENCE: 252
catgaacccg ggaggtggag gctacagtaa gctattatct cccccctgta ct
#ccagcctg 60
ggtgacagag cgagccccca gactcaaaaa ataaaaataa aaaccctgaa ta
#ttttcctt 120
tt
#
#
# 122
<210> SEQ ID NO 253
<211> LENGTH: 53
<212> TYPE: DNA
<213> ORGANISM: Artificial sequence
<220> FEATURE:
<223> OTHER INFORMATION: Synthetic oligonucleotide
<400> SEQUENCE: 253
catgctcata agaaagacag gaaatggttt ggcagg
#tctt ccagtggaca taa
#53
<210> SEQ ID NO 254
<211> LENGTH: 63
<212> TYPE: DNA
<213> ORGANISM: Artificial sequence
<220> FEATURE:
<223> OTHER INFORMATION: Synthetic oligonucleotide
<400> SEQUENCE: 254
catggatgat tatcatttta catcaattaa gtccttactg gcctatgatt ta
#gggctaac 60
tac
#
#
# 63
<210> SEQ ID NO 255
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 255
catgtgccct caaa
#
#
# 14
<210> SEQ ID NO 256
<211> LENGTH: 40
<212> TYPE: DNA
<213> ORGANISM: Artificial sequence
<220> FEATURE:
<223> OTHER INFORMATION: Synthetic oligonucleotide
<400> SEQUENCE: 256
catgatgccc acgggtacca ctagaagcgg aagcgagagg
#
# 40
<210> SEQ ID NO 257
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 257
catgaatgga aaaa
#
#
# 14
<210> SEQ ID NO 258
<211> LENGTH: 137
<212> TYPE: DNA
<213> ORGANISM: Artificial sequence
<220> FEATURE:
<223> OTHER INFORMATION: Synthetic oligonucleotide
<400> SEQUENCE: 258
catgggtcaa gtcggtcctg agagatgggc gagcgccgtt ccgaagggac gg
#gcgatggc 60
ctccagccct tagagccaat ccttatcccg aagttacgga tccggcttgc cg
#acttccct 120
tacctacatt gactcgg
#
#
# 137
<210> SEQ ID NO 259
<211> LENGTH: 79
<212> TYPE: DNA
<213> ORGANISM: Artificial sequence
<220> FEATURE:
<223> OTHER INFORMATION: Synthetic oligonucleotide
<400> SEQUENCE: 259
catgcattgc cttcatttat tgtatttcaa atcactgtac atttactttt gt
#gaaaacac 60
tacctgcatt ttctagtac
#
#
# 79
<210> SEQ ID NO 260
<211> LENGTH: 43
<212> TYPE: DNA
<213> ORGANISM: Artificial sequence
<220> FEATURE:
<223> OTHER INFORMATION: Synthetic oligonucleotide
<400> SEQUENCE: 260
catgatccaa aggaacaagc atcaagcacg cagcaatgca gct
#
# 43
<210> SEQ ID NO 261
<211> LENGTH: 67
<212> TYPE: DNA
<213> ORGANISM: Artificial sequence
<220> FEATURE:
<223> OTHER INFORMATION: Synthetic oligonucleotide
<400> SEQUENCE: 261
catgtgttaa tgttgacaat ttagaaaaca cagacaagtg aaagaaaaga ga
#aagaatga 60
gaaaagg
#
#
# 67
<210> SEQ ID NO 262
<211> LENGTH: 26
<212> TYPE: DNA
<213> ORGANISM: Artificial sequence
<220> FEATURE:
<223> OTHER INFORMATION: Synthetic oligonucleotide
<400> SEQUENCE: 262
catggacaag agcgaaactc cgtctc
#
# 26
<210> SEQ ID NO 263
<211> LENGTH: 46
<212> TYPE: DNA
<213> ORGANISM: Artificial sequence
<220> FEATURE:
<223> OTHER INFORMATION: Synthetic oligonucleotide
<400> SEQUENCE: 263
catgtgttaa tgttgaatca aaacttttct ttaggtgtgc atatat
# 46
<210> SEQ ID NO 264
<211> LENGTH: 35
<212> TYPE: DNA
<213> ORGANISM: Artificial sequence
<220> FEATURE:
<223> OTHER INFORMATION: Synthetic oligonucleotide
<400> SEQUENCE: 264
catgtcctgc agaataataa agtcatcaat acctc
#
# 35
<210> SEQ ID NO 265
<211> LENGTH: 32
<212> TYPE: DNA
<213> ORGANISM: Artificial sequence
<220> FEATURE:
<223> OTHER INFORMATION: Synthetic oligonucleotide
<400> SEQUENCE: 265
catgacgcag gaagcagctg aaagagctat tg
#
# 32
<210> SEQ ID NO 266
<211> LENGTH: 255
<212> TYPE: DNA
<213> ORGANISM: Artificial sequence
<220> FEATURE:
<223> OTHER INFORMATION: Synthetic oligonucleotide
<400> SEQUENCE: 266
catgggacca cgggtgacgg ggaatcaggg ttcgattccg gagagggagc ct
#gagaaacg 60
gctaccacat ccaaggtagg cagcaggcgc gcaaattacc cactcccgac cc
#ggggaggt 120
agtgacgaaa aataacaata caggactctt tcgaggccct gtaattggaa tg
#agtccact 180
ttaaatcctt taacgaggat ccattggagg gcaagtctgg tgccagcagc cg
#cggtaatt 240
ccagctccaa tagcg
#
#
# 255
<210> SEQ ID NO 267
<211> LENGTH: 25
<212> TYPE: DNA
<213> ORGANISM: Artificial sequence
<220> FEATURE:
<223> OTHER INFORMATION: Synthetic oligonucleotide
<400> SEQUENCE: 267
catgtagtac accctctaaa tcacc
#
# 25
<210> SEQ ID NO 268
<211> LENGTH: 92
<212> TYPE: DNA
<213> ORGANISM: Artificial sequence
<220> FEATURE:
<223> OTHER INFORMATION: Synthetic oligonucleotide
<400> SEQUENCE: 268
catgttctca taggagatct ccacaggggc tggacggttc attatggcaa at
#aaaaagtg 60
tgagcgtgcg tgtgagtgtg atggggaaag tg
#
# 92
<210> SEQ ID NO 269
<211> LENGTH: 59
<212> TYPE: DNA
<213> ORGANISM: Artificial sequence
<220> FEATURE:
<223> OTHER INFORMATION: Synthetic oligonucleotide
<400> SEQUENCE: 269
catggagagg tggaagacag aagtacggga aggcgaagaa aagaatagag aa
#gataggg 59
<210> SEQ ID NO 270
<211> LENGTH: 25
<212> TYPE: DNA
<213> ORGANISM: Artificial sequence
<220> FEATURE:
<223> OTHER INFORMATION: Synthetic oligonucleotide
<400> SEQUENCE: 270
catgaagaat gcaggaaaga gatcg
#
# 25
<210> SEQ ID NO 271
<211> LENGTH: 126
<212> TYPE: DNA
<213> ORGANISM: Artificial sequence
<220> FEATURE:
<223> OTHER INFORMATION: Synthetic oligonucleotide
<400> SEQUENCE: 271
catggagatt atctttgttt tagttttcct tctagcatat tattgtaaaa tt
#aatagtat 60
atagaataac agtttcttgc atactacagc atttttgtct gatttttttt cg
#tagtagcc 120
atagcc
#
#
# 126
<210> SEQ ID NO 272
<211> LENGTH: 255
<212> TYPE: DNA
<213> ORGANISM: Artificial sequence
<220> FEATURE:
<223> OTHER INFORMATION: Synthetic oligonucleotide
<400> SEQUENCE: 272
catgacccag ggagggtggt cagcagcgca gcctgcccag gctgggatct cc
#ctttggtc 60
atagccgtgt ttccatatac ccctccactc accctcagag gaggaacgga tg
#gaagccac 120
cagcataaat aaagggaaca cagaagaaca atgtcaccaa agtgcaggtg ca
#aagcccaa 180
agcagccccc tacctctgcc agcccagacc cgccactaaa ttctagagga gg
#gtgtctct 240
caggtcacag tactt
#
#
# 255
<210> SEQ ID NO 273
<211> LENGTH: 39
<212> TYPE: DNA
<213> ORGANISM: Artificial sequence
<220> FEATURE:
<223> OTHER INFORMATION: Synthetic oligonucleotide
<400> SEQUENCE: 273
catgtgaggg aaaatgaaag ctactcatag cgggggcct
#
# 39
<210> SEQ ID NO 274
<211> LENGTH: 96
<212> TYPE: DNA
<213> ORGANISM: Artificial sequence
<220> FEATURE:
<223> OTHER INFORMATION: Synthetic oligonucleotide
<400> SEQUENCE: 274
catgagaaaa cagtatgccc attaatttac tttgtgttca aaataaaatt cc
#aactgctg 60
ttgggggggg ccccagaaac gaaaaataac cccccc
#
# 96
<210> SEQ ID NO 275
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 275
catgctcata aaga
#
#
# 14
<210> SEQ ID NO 276
<211> LENGTH: 69
<212> TYPE: DNA
<213> ORGANISM: Artificial sequence
<220> FEATURE:
<223> OTHER INFORMATION: Synthetic oligonucleotide
<400> SEQUENCE: 276
catgtatgaa aacagaagac aaaattgtga gccagagtcg acaaatgaaa ta
#aattaccc 60
cctcctccg
#
#
# 69
<210> SEQ ID NO 277
<211> LENGTH: 102
<212> TYPE: DNA
<213> ORGANISM: Artificial sequence
<220> FEATURE:
<223> OTHER INFORMATION: Synthetic oligonucleotide
<400> SEQUENCE: 277
catgaggttg acagtgtaaa cctgctctaa gttttcactt tgcactgtgt ac
#caaaatga 60
aactgcttat ttaggaaaat aaaaatatct tttgtgttgg ac
#
# 102
<210> SEQ ID NO 278
<211> LENGTH: 95
<212> TYPE: DNA
<213> ORGANISM: Artificial sequence
<220> FEATURE:
<223> OTHER INFORMATION: Synthetic oligonucleotide
<400> SEQUENCE: 278
catgaggttg acagtgaggg taataaaagt aaaaggaact aattttggaa aa
#gctaagac 60
agaagaaaag gaccttaaga agctaaaaaa gcagg
#
# 95
<210> SEQ ID NO 279
<211> LENGTH: 40
<212> TYPE: DNA
<213> ORGANISM: Artificial sequence
<220> FEATURE:
<223> OTHER INFORMATION: Synthetic oligonucleotide
<400> SEQUENCE: 279
catggtaact gattcttaac agattcatat atgtatctgt
#
# 40
<210> SEQ ID NO 280
<211> LENGTH: 126
<212> TYPE: DNA
<213> ORGANISM: Artificial sequence
<220> FEATURE:
<223> OTHER INFORMATION: Synthetic oligonucleotide
<400> SEQUENCE: 280
catgttaatt acagcacatt aaaatggtgg tttacattac aaataagcct gt
#aagtttaa 60
atatactagt gttataaccc aatgtacaga cgttctttat acaatacata ca
#attatcag 120
gaatgc
#
#
# 126
<210> SEQ ID NO 281
<211> LENGTH: 31
<212> TYPE: DNA
<213> ORGANISM: Artificial sequence
<220> FEATURE:
<223> OTHER INFORMATION: Synthetic oligonucleotide
<400> SEQUENCE: 281
catggaggac cattaaaatg tgacaccttt c
#
# 31
<210> SEQ ID NO 282
<211> LENGTH: 15
<212> TYPE: DNA
<213> ORGANISM: Artificial sequence
<220> FEATURE:
<223> OTHER INFORMATION: Synthetic oligonucleotide
<400> SEQUENCE: 282
catgtgccct cagag
#
#
# 15
<210> SEQ ID NO 283
<211> LENGTH: 44
<212> TYPE: DNA
<213> ORGANISM: Artificial sequence
<220> FEATURE:
<223> OTHER INFORMATION: Synthetic oligonucleotide
<400> SEQUENCE: 283
catgtgccct cagattccgc accaataaag ccttcaaact cccg
#
# 44
<210> SEQ ID NO 284
<211> LENGTH: 38
<212> TYPE: DNA
<213> ORGANISM: Artificial sequence
<220> FEATURE:
<223> OTHER INFORMATION: Synthetic oligonucleotide
<400> SEQUENCE: 284
catgtaaagt ggaataaaag ttttatgaat ggactttt
#
# 38
<210> SEQ ID NO 285
<211> LENGTH: 175
<212> TYPE: DNA
<213> ORGANISM: Artificial sequence
<220> FEATURE:
<223> OTHER INFORMATION: Synthetic oligonucleotide
<400> SEQUENCE: 285
catgcttaat cactgcaatt ttaagctact gtacacagga atgaaaaggt ta
#tagaaaag 60
tgccatagca acagtgcctt aagaaaggag ataaagagga gccttaaaaa aa
#tggataaa 120
atcagaattt cagaaggaaa tggaaacaca cgggaaatga aaaacatttc tc
#tgc 175
<210> SEQ ID NO 286
<211> LENGTH: 80
<212> TYPE: DNA
<213> ORGANISM: Artificial sequence
<220> FEATURE:
<223> OTHER INFORMATION: Synthetic oligonucleotide
<400> SEQUENCE: 286
catgatggct cttgatggca gttagctctt attgctgaaa ataaaataaa aa
#taaataaa 60
aaagcggccg ctctagatag
#
#
# 80
<210> SEQ ID NO 287
<211> LENGTH: 101
<212> TYPE: DNA
<213> ORGANISM: Artificial sequence
<220> FEATURE:
<223> OTHER INFORMATION: Synthetic oligonucleotide
<400> SEQUENCE: 287
catggagcac ttggcgtgcc ataatgatgg gaatagctgt cagctgtaag at
#caccattg 60
ctgactggac aactgcaata aatttgacgg gtgtttctct t
#
# 101
<210> SEQ ID NO 288
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 288
catggttcag gaaa
#
#
# 14
<210> SEQ ID NO 289
<211> LENGTH: 25
<212> TYPE: DNA
<213> ORGANISM: Artificial sequence
<220> FEATURE:
<223> OTHER INFORMATION: Synthetic oligonucleotide
<400> SEQUENCE: 289
catgtggccc agtatgtggt acggg
#
# 25
<210> SEQ ID NO 290
<211> LENGTH: 16
<212> TYPE: DNA
<213> ORGANISM: Artificial sequence
<220> FEATURE:
<223> OTHER INFORMATION: Synthetic oligonucleotide
<400> SEQUENCE: 290
catggacagt agatgg
#
#
# 16
<210> SEQ ID NO 291
<211> LENGTH: 80
<212> TYPE: DNA
<213> ORGANISM: Artificial sequence
<220> FEATURE:
<223> OTHER INFORMATION: Synthetic oligonucleotide
<400> SEQUENCE: 291
catgagggag gcagaggttg cagtgagccg agatcgaacc atcgcattcc ag
#ccttggcg 60
acagaatgag acactgtttc
#
#
# 80
<210> SEQ ID NO 292
<211> LENGTH: 100
<212> TYPE: DNA
<213> ORGANISM: Artificial sequence
<220> FEATURE:
<223> OTHER INFORMATION: Synthetic oligonucleotide
<400> SEQUENCE: 292
catgagtgag aaggcaggtt gtgcgggtgt tgaccgatgt atcttttcct ta
#aagttatt 60
ataataatgg gtaatttgtc aataaagcat tcctttgggg
#
# 100
<210> SEQ ID NO 293
<211> LENGTH: 70
<212> TYPE: DNA
<213> ORGANISM: Artificial sequence
<220> FEATURE:
<223> OTHER INFORMATION: Synthetic oligonucleotide
<400> SEQUENCE: 293
catgggcctg gcactgcaca ctcagttctg ctctaagaag ctgcaataaa gt
#ttttttaa 60
gtcactttgg
#
#
# 70
<210> SEQ ID NO 294
<211> LENGTH: 44
<212> TYPE: DNA
<213> ORGANISM: Artificial sequence
<220> FEATURE:
<223> OTHER INFORMATION: Synthetic oligonucleotide
<400> SEQUENCE: 294
catggttaaa tgcatttcct aatttgagat cccctaaacc ctgg
#
# 44
<210> SEQ ID NO 295
<211> LENGTH: 91
<212> TYPE: DNA
<213> ORGANISM: Artificial sequence
<220> FEATURE:
<223> OTHER INFORMATION: Synthetic oligonucleotide
<400> SEQUENCE: 295
catgtgaaag ttttatgctc tgcatttgcc cctggtgatt ttaaaatttg tt
#tgcctttt 60
taaagctata ttaaaaatgt attgttgaat c
#
# 91
<210> SEQ ID NO 296
<211> LENGTH: 96
<212> TYPE: DNA
<213> ORGANISM: Artificial sequence
<220> FEATURE:
<223> OTHER INFORMATION: Synthetic oligonucleotide
<400> SEQUENCE: 296
catgatctta cctgctcgaa ttcaagcttg taacgatgta cgggagatgg cc
#gttcttag 60
ttggtggagc gatttgtctg gttaattccg ataacg
#
# 96
<210> SEQ ID NO 297
<211> LENGTH: 47
<212> TYPE: DNA
<213> ORGANISM: Artificial sequence
<220> FEATURE:
<223> OTHER INFORMATION: Synthetic oligonucleotide
<400> SEQUENCE: 297
catgcatttg tcttttcata aaaaaattac gtaaaaatgc ccccagg
# 47
<210> SEQ ID NO 298
<211> LENGTH: 54
<212> TYPE: DNA
<213> ORGANISM: Artificial sequence
<220> FEATURE:
<223> OTHER INFORMATION: Synthetic oligonucleotide
<400> SEQUENCE: 298
catgctgcac taccaggttc tagcaagagg agaagaaagg aggaaaccac ag
#gg 54
<210> SEQ ID NO 299
<211> LENGTH: 63
<212> TYPE: DNA
<213> ORGANISM: Artificial sequence
<220> FEATURE:
<223> OTHER INFORMATION: Synthetic oligonucleotide
<400> SEQUENCE: 299
catgtataac aaaagcttat tttcattatt attataataa acagttaatg gt
#tgagacat 60
ttt
#
#
# 63
<210> SEQ ID NO 300
<211> LENGTH: 67
<212> TYPE: DNA
<213> ORGANISM: Artificial sequence
<220> FEATURE:
<223> OTHER INFORMATION: Synthetic oligonucleotide
<400> SEQUENCE: 300
catgacagca acctggatgg aactggaggc tgttatctta agtgaaataa ct
#tagaagta 60
gaaagtc
#
#
# 67
<210> SEQ ID NO 301
<211> LENGTH: 34
<212> TYPE: DNA
<213> ORGANISM: Artificial sequence
<220> FEATURE:
<223> OTHER INFORMATION: Synthetic oligonucleotide
<400> SEQUENCE: 301
catgacctgc agaataataa agtcatcaat accg
#
# 34
<210> SEQ ID NO 302
<211> LENGTH: 83
<212> TYPE: DNA
<213> ORGANISM: Artificial sequence
<220> FEATURE:
<223> OTHER INFORMATION: Synthetic oligonucleotide
<400> SEQUENCE: 302
catgcgacga cgctatgaac gcttggccgc cacaagccag ttatccctgt gg
#taactttt 60
ctgacacctc ctgcttaaaa ccc
#
# 83
<210> SEQ ID NO 303
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 303
catgctcaaa agga
#
#
# 14
<210> SEQ ID NO 304
<211> LENGTH: 23
<212> TYPE: DNA
<213> ORGANISM: Artificial sequence
<220> FEATURE:
<223> OTHER INFORMATION: Synthetic oligonucleotide
<400> SEQUENCE: 304
catgtaagag gacatgagga agg
#
# 23
<210> SEQ ID NO 305
<211> LENGTH: 101
<212> TYPE: DNA
<213> ORGANISM: Artificial sequence
<220> FEATURE:
<223> OTHER INFORMATION: Synthetic oligonucleotide
<400> SEQUENCE: 305
catgtgacca tcatagccac catcaccctc cttaacctct acttctacct ac
#gcctaatc 60
tactccacct caatcacact actccccata tctaacaacg t
#
# 101
<210> SEQ ID NO 306
<211> LENGTH: 14
<212> TYPE: DNA
<213> ORGANISM: Artificial sequence
<220> FEATURE:
<223> OTHER INFORMATION: Synthetic oligonucleotide
<400> SEQUENCE: 306
catgttactt gtcg
#
#
# 14
<210> SEQ ID NO 307
<211> LENGTH: 105
<212> TYPE: DNA
<213> ORGANISM: Artificial sequence
<220> FEATURE:
<223> OTHER INFORMATION: Synthetic oligonucleotide
<400> SEQUENCE: 307
catgcttttt gtgctcccct ttgcctaaac cgtatggcct cccgtgcatc tg
#tactcacc 60
ctgtacgaca aacacattac attattaaat gtttctcaaa gatgg
# 105
<210> SEQ ID NO 308
<211> LENGTH: 67
<212> TYPE: DNA
<213> ORGANISM: Artificial sequence
<220> FEATURE:
<223> OTHER INFORMATION: Synthetic oligonucleotide
<400> SEQUENCE: 308
catgtgctgg gtgggttcag agggcaattt ctcttttatg tgtacatatg ct
#aaataaac 60
ataattt
#
#
# 67
<210> SEQ ID NO 309
<211> LENGTH: 113
<212> TYPE: DNA
<213> ORGANISM: Artificial sequence
<220> FEATURE:
<223> OTHER INFORMATION: Synthetic oligonucleotide
<400> SEQUENCE: 309
catgcctgta gttccagtta ctggagtgga gtggctgagt taggaagaca gg
#aggatagc 60
ttgattctaa gaggttgagt ctagcctgga caatatagtg agatcctgtc tg
#t 113
<210> SEQ ID NO 310
<211> LENGTH: 25
<212> TYPE: DNA
<213> ORGANISM: Artificial sequence
<220> FEATURE:
<223> OTHER INFORMATION: Synthetic oligonucleotide
<400> SEQUENCE: 310
catgaaagcg gccgctctag atagt
#
# 25
<210> SEQ ID NO 311
<211> LENGTH: 55
<212> TYPE: DNA
<213> ORGANISM: Artificial sequence
<220> FEATURE:
<223> OTHER INFORMATION: Synthetic oligonucleotide
<400> SEQUENCE: 311
catgtacccg cctcctttct gttttatttt tgaggaaata aaataaccaa gt
#gct 55
<210> SEQ ID NO 312
<211> LENGTH: 30
<212> TYPE: DNA
<213> ORGANISM: Artificial sequence
<220> FEATURE:
<223> OTHER INFORMATION: Synthetic oligonucleotide
<400> SEQUENCE: 312
catgacacag caagcggccg ctctagatag
#
# 30
<210> SEQ ID NO 313
<211> LENGTH: 15
<212> TYPE: DNA
<213> ORGANISM: Artificial sequence
<220> FEATURE:
<223> OTHER INFORMATION: Synthetic oligonucleotide
<400> SEQUENCE: 313
atctagagcg gccgc
#
#
# 15
<210> SEQ ID NO 314
<211> LENGTH: 15
<212> TYPE: DNA
<213> ORGANISM: Artificial sequence
<220> FEATURE:
<223> OTHER INFORMATION: Synthetic oligonucleotide
<400> SEQUENCE: 314
atctagagcg gccgc
#
#
# 15
<210> SEQ ID NO 315
<211> LENGTH: 20
<212> TYPE: DNA
<213> ORGANISM: Artificial sequence
<220> FEATURE:
<223> OTHER INFORMATION: Synthetic oligonucleotide
<400> SEQUENCE: 315
actatctaga gcggccgctt
#
#
# 20

What is claimed is:

1. A microarray for measuring gene expression characteristics of hematopoietic cells comprising at least 5 polynucleotides having distinct sequences selected from the group consisting of SEQ ID NOS: 1-244.

2. The microarray according to claim 1 wherein the microarray comprises at least 9 polynucleotides having distinct sequences selected from the group consisting of SEQ ID NOS: 1-244.

3. The microarray according to claim 1 wherein the microarray comprises at least 15 polynucleotides having distinct sequences selected from the group consisting of SEQ ID NOS: 1-244.

4. The microarray according to claim 3 wherein the microarray comprises at least 25 polynucleotides having distinct sequences selected from the group consisting of SEQ ID NOS: 1-244.

5. The microarray according to claim 4 wherein the microarray comprises at least 50 polynucleotides having distinct sequences selected from the group consisting of SEQ ID NOS: 1-244.

6. A method of diagnosing an abnormality associated with hematopoiesis comprising the following steps:
(a) contacting a cell sample nucleic acid with a microarray according to claim 1 under conditions suitable for hybridization;
(b) detecting said hybridization; and
(c) diagnosing a hematopoietic abnormality based on the results of detecting said hybridization.

7. The method according to claim 6 wherein said abnormality is leukemia.

8. A method of treating an abnormality associated with hematopoiesis comprising the following steps:
(a) obtaining a polynucleotide comprising a sequence selected from the group consisting of SEQ ID NOS: 1-244 in an amount effective to treat a condition associated with hematopoiesis; and
(b) delivering said polynucleotide to a mammal in need of such treatment.

9. The method according to claim 8 wherein said abnormality is a disease.

10. The method according to claim 8 wherein said polynucleotide is delivered to said mammal in the form of a pharmaceutical composition.

11. The method according to claim 8 wherein said hematopoietic cells are normal.

12. A method for diagnosing myeloid leukemia in a patient, comprising the steps of:
(a) obtaining a cell sample from said patient,
(b) detecting the expression of one or more of the genes comprising a sequence selected from the group consisting of SEQ ID NO: 1-244 in said sample; and
(c) comparing said expression in step (b) with the expression of said one or more genes in a cell sample obtained from a subject not having leukemia,
wherein a change in expression of said one or more genes is diagnostic of said patient having myeloid leukemia.

13. The method according to claim 12, wherein said sample is a tissue sample selected from the group consisting of bone marrow, serum, and peripheral blood cells.
